(19) United States
(12) Patent Application Publication
(10) Pub. No.: US 2025/0058274 A1
(43) Pub. Date: Feb. 20, 2025

(54) PROCESS AND SYSTEM FOR REMOVING IMPURITIES FROM A GAS STREAM

(71) Applicant: LanzaTech, Inc., Skokie, IL (US)

(72) Inventors: Richard Russell Rosin, Glencoe, IL (US); Yen-Lu Yu, Buffalo Grove, IL (US); Sean Alex Rollag, Chicago, IL (US); Taylor Craig Schulz, Chicago, IL (US)

(21) Appl. No.: 18/651,291

(22) Filed: Apr. 30, 2024

Related U.S. Application Data
(60) Provisional application No. 63/582,196, filed on Sep. 12, 2023.
(60) Provisional application No. 63/519,688, filed on Aug. 15, 2023.

(51) Int. Cl. B01D 53/75; B01D 53/00; B01D 53/02; B01D 53/86; B01J 23/44; B01J 23/72; C12M 1/00

(52) U.S. Cl. CPC B01D 53/75; B01D 53/005; B01D 53/02; B01D 53/8603; B01D 53/8621; B01J 23/44; B01J 23/72; C12M 43/00; B01D 2253/102; B01D 2253/104; B01D 2253/1124; B01D 2255/1023; B01D 2255/20761; B01D 2257/308; B01D 2257/408; B01D 2257/702; B01D 2258/025

(57) ABSTRACT

A process and system for producing a fermentable gas stream from a gas source that contains one or more impurity which may be harmful to the fermentation process is provided. To produce the fermentable gas stream, the gas stream is passed through a specifically ordered series of removal beds. The removal beds remove and/or convert various impurities found in the gas stream which may have harmful effects on downstream removal beds and/or inhibitory effects on downstream gas fermenting microorganisms. At least a portion of the fermentable gas stream may be capable of being passed to a bioreactor, which contains gas fermenting microorganisms.

### CROSS REFERENCE TO RELATED APPLICATIONS

This application claims the benefit of U.S. Provisional Patent Application Nos. 63/519,688 filed on Aug. 15, 2023, and 63/582,196 filed on Sep. 12, 2023, the entirety of which are incorporated herein by references.

### FIELD

This invention relates to systems and processes for removing impurities from a gas stream. In particular, the invention relates to the removal of impurities from a gas stream that may be harmful to subsequent removal beds and/or C1-fixing microorganisms in a downstream fermentation process.

### BACKGROUND

The following discussion is provided to aid the reader in understanding the disclosure and is not admitted to describe or constitute prior art thereto.

Mitigation of impending climate change requires drastic changes in manufacturing and greater reliance on biotechnology. Sustainable sources of fuels and chemicals are currently insufficient to significantly displace dependence on fossil carbon. Biotechnology harnesses the power of biology to create new products in a way that improves the quality of life and the environment, Gas fermentation is emerging as a powerful biotechnological advancement as an alternative platform for the biological fixation of such gases such as CH₄, CO, CO₂, and/or H₂ into sustainable fuels and chemicals. In particular, gas fermentation technology can utilize a wide range of feedstocks including gasified carbon-containing matter such as municipal solid waste or agricultural waste, or industrial waste gases such as off-gases from steel manufacturing, petroleum refineries, and petrochemical processes to produce ethanol, aviation fuel, chemicals, and a variety of other products. Gas fermentation alone could displace 30% of crude oil use and reduce global CO₂ emissions by 10%. As with any disruptive technology, many technical challenges must be overcome before this potential is fully achieved. The science of scale-up production and the reduction of obstacles for continued commercialization of gas fermentation are advanced by this disclosure.

Gas fermentation processes can be used to generate target materials from gas substrates or other input materials, particularly carbon-based materials. For example, particular biological systems can be used to perform gas fermentation. Such processes and systems are alternatives to traditional processes with an important benefit of aiding in combating climate change. C1-Fixing microorganisms that fix carbon dioxide (CO₂) and/or carbon monoxide (CO) can ease the effect of dependence on fossil carbon as these microorganisms they can convert gaseous carbon into useful fuels and chemicals.

Industrial processes can output gases that have significant amounts of carbon-based materials. Chemical processors and oil refiners generally view flaring and venting carbon rich sources to the atmosphere or otherwise discarding them as traditional standard operations. Currently, the primary alternative available to chemical processors and oil refiners is to engage in some form of carbon capture and sequestration (“CCS”). CCS can include finding permanent underground storage such as depleted oil wells or sealed saline aquifers to permanently store the gaseous carbon.

Furthermore, a vast amount of “above-ground” carbon exists today that may be recycled from carbon in waste into carbon in newly produced chemicals by gas fermentation processes. Waste may be gasified into syngas which in turn becomes the feedstock to a gas fermentation process to generate a newly produced chemical thus recycling the carbon. This cycle is carbon capture and utilisation (“CCU”) as opposed to CCS.

The wide variety of industries producing these gas streams invariably introduce impurities due to process variables and trace elements in process feedstocks. These impurities can affect downstream conversion performance of gas fermenting microorganisms. For example, mono nitrogenous species such as hydrogen cyanide (HCN), ammonia (NH₃), nitrogen oxide (NOx) and other known enzyme inhibiting gases such as acetylene (C₂H₂), ethylene (C₂H₄), BTEX (benzene, toluene, ethyl benzene, xylene), and oxygen (O₂) can be present. Sulfur compounds in the gas such as hydrogen sulfide (H₂S), carbonyl sulfide (COS), carbon disulfide (CS₂), and sulfur oxide (Sox) compounds such as SO₂ and SO₃ can in turn negatively affect catalyst-based scrubbing systems. The same is true for syngas generated from carbon-containing waste material. Even more variability may be found in syngas.

For many impurity compounds, commercially available removal systems exist; however, these systems have not been used for microbial gas fermentation. Microbial gas fermentation, as the downstream process, is a relatively new alternative to conventional catalytic conversion technologies and requires relatively specific impurity limitations. To ensure successful, noninhibited gas fermentation, treatment of these gases must be completed in order to protect the F1-fixing microorganisms.

There are three central concerns with cleaning gas for gas fermentation, including (1) excessive consumption of the desired reactant compounds for microbial fermentation; (2) reaction to form other undesired compounds which will act as microbial inhibitors; and (3) reduction of the inhibitory compounds in the feed stream to sufficiently low levels to ensure successful, noninhibited gas fermentation.

In industry, situations arise where an existing equipment in use for a process may be revamped to accommodate technology advancements or repurposed for use in another process. Often the goal is to minimize capex by using existing equipment instead of constructing new equipment. Current gas treatment processes may employ three or more heat exchangers, and three, four, or more individual vessels each containing a material to react and or remove an impurity from the source gas. In a revamp situation, often the desired number of vessels and heat exchangers are not available. Therefore, a need exists to consolidate several traditional gas treatment steps into one vessel. However, the challenge is that the consolidated steps would need to operate at the same temperature as they are co-located in the same vessel. Normally, adsorbents require a lower temperature and catalysts require a comparatively elevated temperature. A need exists to select adsorbents and catalysts that perform their function at the same temperature when co-located in the same vessel. As all material co-located in a single vessel which is operated at a common temperature, as little as one heater or heat exchanger is needed.

Accordingly, there remains a need for an invention that strategically treats gas streams from industrial or other processes and or syngas to provide a suitable gas as feedstock for a downstream fermentation process.

### SUMMARY

The disclosure provides a process for removing impurities from an input gas stream to produce a fermentable gas stream comprising:
a. heating the input gas stream to a temperature that is effective for a deoxygenation catalyst and below a reduction temperature of a sulfur guard bed material to generate a heated input gas stream;
b. contacting, in a vessel, the heated input gas stream with:
   i. a hydrolysis catalyst bed to remove hydrogen cyanide to less than 1 ppm hydrogen cyanide and/or carbonyl sulfide to less than 1 ppm carbonyl sulfide;
   ii. an optional sulfur guard bed containing material effective to remove and/or react sulfur-containing compounds located concurrent with or downstream of the hydrolysis bed; and
   iii. a deoxygenation catalyst bed located downstream of the sulfur guard bed;
c. wherein the fermentable gas stream comprises less than 100 ppm oxygen, less than 1 ppm acetylene, and less than 1 ppm hydrogen cyanide.

In an embodiment, the process further comprising contacting, in the vessel, the heated input gas stream with a hydrocarbon removal adsorbent bed located upstream of the hydrolysis catalyst bed or contacting the input gas stream, in a module upstream of the heating and the vessel, with a hydrocarbon removal adsorbent bed.

In an embodiment the hydrolysis catalyst bed and the sulfur guard bed are a physical mixture forming a combined hydrolysis and sulfur guard bed.

In an embodiment the process further comprising passing at least a portion of the fermentable gas stream to a bioreactor, wherein the bioreactor contains a culture comprising a fermentation broth and at least one C1-fixing microorganism.

In an embodiment at least a portion of the input gas stream is a synthesis gas and/or a producer gas.

In an embodiment the process further comprises measuring the concentration of impurities in the input gas stream and/or the fermentable gas stream.

In an embodiment the input gas stream comprises CO, CO₂, H₂, or any combination thereof.

In an embodiment the sulfur guard bed material is zinc oxide or copper and zinc oxide supported on alumina.

In an embodiment the deoxygenation catalyst comprises copper supported on alumina, silica, titania, ceria, lanthana, carbon, silica-alumina, or a zeolite.

In an embodiment the hydrocarbon removal adsorbent is activated carbon.

In an embodiment the hydrolysis bed and the sulfur guard bed are a combined hydrolysis and sulfur guard bed comprising bifunctional zinc oxide on alumina support or copper and zinc oxide on alumina support.

In an embodiment the hydrolysis bed and the sulfur guard bed are a combined hydrolysis and sulfur guard bed comprising bifunctional zinc oxide on alumina support or copper and zinc oxide on alumina support.

The disclosure provides an apparatus comprising:
a. a heating device having a heating device gas inlet and a heating device gas outlet;
b. a vessel having a vessel gas inlet in fluid communication with the heating device gas outlet and a vessel fermentable gas outlet wherein the vessel contains at least three beds comprising:
   i. a hydrolysis catalyst bed comprising alumina;
   ii. an optional sulfur guard bed comprising zinc oxide or copper and zinc oxide on an alumina support located concurrent with or downstream of the hydrolysis bed;
   iii. a deoxygenation catalyst bed located downstream of the sulfur guard bed, wherein the deoxygenation catalyst comprises copper supported on alumina, silica, titania, ceria, lanthana, carbon, silica-alumina, or zeolites; and
c. a bioreactor having a bioreactor gas inlet in fluid communication with the vessel fermentable gas outlet and a bioreactor fermentation broth output wherein the bioreactor comprises at least one C1-fixing microorganism.

In an embodiment, the apparatus further comprises at least one monitoring device in communication with the vessel gas inlet, the vessel fermentable gas outlet, or both.

In an embodiment the vessel further comprises a hydrocarbon removal bed comprising activated charcoal located in the vessel upstream of the hydrolysis catalyst bed.

In an embodiment the apparatus further comprising a hydrocarbon removal module comprising activated charcoal and having a hydrocarbon removal module gas inlet and hydrocarbon removal module gas outlet, the hydrocarbon removal module gas outlet in fluid communication with the heating device gas inlet.

The disclosure provides a process of revamping a gas treatment system comprising:
a. co-locating, in a repurposed vessel,
   i. a hydrolysis catalyst bed to remove hydrogen cyanide to less than 1 ppm hydrogen cyanide and/or carbonyl sulfide to less than 1 ppm carbonyl sulfide;
   ii. an optional sulfur guard bed containing material effective to remove and/or react sulfur-containing compounds located concurrent with or downstream of the hydrolysis bed; and
   iii. a deoxygenation catalyst bed located downstream of the sulfur guard bed.

In an embodiment the process further comprising connecting a repurposed heating device upstream of the repurposed vessel.

In an embodiment, the process further comprising connecting a hydrocarbon removal module comprising activated charcoal upstream of the repurposed heating device.

As stated above, the disclosure provides a process for producing a fermentable gas stream from an input gas stream comprising CO, CO₂, H₂, or a combination thereof, wherein the process comprises passing the input gas stream to a hydrolysis bed, wherein at least one impurity of the gas stream is removed and/or converted to provide a post-hydrolysis gas stream, passing the post-hydrolysis gas stream to sulfur guard bed or an acid gas removal bed, wherein at least one further impurity of the gas stream is removed and/or converted to produce an acid gas depleted stream, passing the acid gas depleted stream to a deoxygenation bed, wherein at least one further impurity is removed and/or converted to produce a fermentable gas stream.

In at least one embodiment, at least one impurity removed is a microorganism inhibitor and/or a catalyst inhibitor.

In embodiments, at least one or more of the impurities removed and/or converted by the hydrolysis bed is carbonyl sulfide (COS) and/or hydrogen cyanide (HCN).

The impurities removed and/or converted by the acid gas removal bed may be selected from the group consisting of carbon dioxide (CO₂), hydrogen sulfide (H₂S), and hydrogen cyanide (HCN).

In embodiments, at least one or more of the impurities removed and/or converted by the deoxygenation bed is oxygen (O₂) and/or acetylene (C₂H₂).

In certain instances, the hydrolysis bed is bypassed, and the input gas stream is delivered to the acid gas removal bed.

The process may further include a catalytic hydrogenation bed. In embodiments utilizing a catalytic hydrogenation bed, the acid gas depleted stream is passed to the catalytic hydrogenation bed, prior to being passed to the deoxygenation bed, wherein at least one impurity from the acid gas depleted stream is removed and/or converted prior to being passed to the deoxygenation bed. At least one impurity removed and/or converted by the catalytic hydrogenation bed is acetylene (C₂H₂).

The process may include at least one additional bed selected from the group comprising: particulate removal bed, chloride removal bed, tar removal bed, hydrogen cyanide removal bed, additional acid gas removal bed, temperature module, and pressure module.

In particular instances, the additional acid gas removal bed is a pressure swing adsorption (PSA) bed or a temperature swing adsorption bed (TSA).

In embodiments, the process includes monitoring devices for measuring the level of impurities present in the gas stream. The one or more monitoring devices may be placed before and/or after one or more bed. In certain instances, the process may be capable of bypassing one or more bed as a function of the level of one or more impurity in the gas stream.

The process may include a hydrogen cyanide removal bed capable of receiving the post-deoxygenation gas stream. The hydrogen cyanide removal bed may remove at least a portion of the hydrogen cyanide from the gas stream prior to passing the gas stream to the bioreactor.

The impurity levels may be reduced to predetermined levels prior to being passed to the bioreactor, such that the gas stream is fermentable. In embodiments, the predetermined level of impurities comprises no more than one hundred parts per million (100 ppm) oxygen (O₂), one part per million (1 ppm) hydrogen cyanide (HCN), and one part per million (1 ppm) acetylene (C₂H₂). In certain instances, the predetermined level of impurities comprises no more than one hundred parts per billion (100 ppb) hydrogen cyanide (HCN).

The bioreactor may contain a culture comprising a fermentation broth and one or more microorganisms. In embodiments, the one or more microorganisms is a C1-fixing or a carboxydotrophic bacterium.

The process may be capable of sending the treated gas stream to a carbon capture means instead of, or prior to, the treated gas stream being passed to the bioreactor.

The particular embodiments, the process is capable of receiving gas streams from one or more sources. At least a portion of the gas stream may be derived from an industrial source. Additionally, at least a portion of the gas stream may be a synthesis gas. Furthermore, at least a portion of the gas stream may be a producer gas.

In embodiments, the invention provides a process for producing a fermentable gas stream, wherein the process comprises treating a gas stream comprising CO, CO₂, or H₂ in a gas treatment process to remove one or more undesired impurity from the gas stream, wherein the step of treating the gas stream comprises passing the gas stream to a hydrolysis bed, wherein at least one impurity of the gas stream is converted to provide a post-hydrolysis stream, passing the post-hydrolysis stream to an acid gas removal bed, wherein at least one further impurity of the stream is removed to provide an acid gas depleted stream, and passing the acid gas depleted stream to a deoxygenation bed, wherein at least one further impurity is converted to provide a fermentable gas stream.

The fermentable gas stream may comprise depleted levels of oxygen (O₂), hydrogen cyanide (HCN), and acetylene (C₂H₂) compared to the input gas stream prior to being passed through the treatment process.

In one embodiment, the fermentable gaseous stream comprises less than one-hundred parts per million (100 ppm) oxygen (O₂).

In one embodiment, the fermentable gaseous stream comprises less than one part per million (1 ppm) hydrogen cyanide (HCN). The fermentable gaseous substrate may comprise less than one hundred parts per billion (100 ppb) hydrogen cyanide (HCN).

In one embodiment, the fermentable gaseous stream comprises less than one part per million (1 ppm) acetylene (C₂H₂).

In various embodiments, the process utilizes one or more specialized catalysts to produce a fermentable gas stream from an input gas stream. The specialized catalyst may be used to reduce the oxygen to less than 100 ppm, acetylene to less than 1 ppm, and the hydrogen cyanide to less than 1 ppm. In certain instances, the specialized catalyst comprises reduced copper metal on a high surface area catalyst such as silica, alumina, titania, ceria, lanthana, silica-alumina, carbon, or many other materials known to those skilled in the art. In certain instances, the specialized catalyst used is copper (I) supported on alumina. In certain instances, the specialized catalyst comprises sulfided copper (I) supported on alumina, such that it is tolerant to sulfur. In certain instances, the specialized catalyst comprises copper (II) supported on alumina. In certain instances, the specialized catalyst comprises sulfided copper (II) supported on alumina, such that it is tolerant to sulfur. The specialized catalyst may comprise sulfided copper supported on alumina when treating an input gas stream with high sulfur content.

In various embodiments, the process receives an input stream comprising various impurities at various levels. In certain instances, the input gas stream comprises oxygen up to 7000 ppm, acetylene up to 700 ppm, and hydrogen cyanide up to 60 ppm, which may represent a gas received from a steel mill. In certain instances, the input stream comprises oxygen up to 10,000 ppm, acetylene up to 1500 ppm, and hydrogen cyanide up to 500 ppm, which may represent a gas stream from a gasification process (biomass or municipal solid waste) or treated coke oven gas. The input gas stream may comprise oxygen up to 7000 ppm, acetylene up to 700 ppm, and hydrogen cyanide up to 60 ppm. The input gas stream may comprise oxygen up to 10000 ppm, acetylene up to 1500 ppm, and hydrogen cyanide up to 500 ppm. The input gas stream may comprise oxygen up to 7000 ppm, acetylene up to 700 ppm, and hydrogen cyanide up to 60 ppm, or the input gas stream may comprise oxygen up to 10000 ppm, acetylene up to 1500 ppm, and hydrogen cyanide up to 500 ppm.

The process may consume less than 10 percent of the carbon monoxide in the input gas stream. The process may, in certain instances, be conducted under pressure. For example, the process may be carried out at a pressure of at least 138 kPag.

At least a portion of the fermentable gas stream may be provided to a bioreactor containing a culture of C1-fixing microorganisms. The C1-fixing microorganism may be a carboxydotrophic bacterium. The carboxydotrophic bacterium may be selected from the group comprising Moorella, Clostridium, Ruminococcus, Acetobacterium, Eubacterium, Butyribacterium, Oxobacter, Methanosarcina, and Desulfotomaculum.

The carboxydotrophic bacterium may be Clostridium autoethanogenum.

In certain instances, the industrial source may be selected from the group consisting of ferrous metal products manufacturing, such as a steel mill manufacturing, non-ferrous products manufacturing, petroleum refining, coal gasification, electric power production, carbon black production, ammonia production, methanol production, and coke manufacturing.

In certain instances, the synthesis gas source is selected from the group consisting of gasification of coal or refinery residues, gasification of biomass or lignocellulosic material, reforming of natural gas, and gasification of municipal solid waste or industrial solid waste.

The disclosure provides a process for removing impurities from an input gas stream to produce a fermentable gas stream comprising:
a. heating the input gas stream to a temperature that is effective for a deoxygenation catalyst and below a reduction temperature of a sulfur guard bed material to generate a heated input gas stream;
b. contacting, in a first vessel, the heated input gas stream with:
   i. a hydrolysis catalyst bed to remove hydrogen cyanide to less than 1 ppm hydrogen cyanide and/or carbonyl sulfide to less than 1 ppm carbonyl sulfide;
   ii. a first sulfur guard bed containing material effective to remove and/or react sulfur-containing compounds, the first sulfur guard bed located concurrent with or downstream of the hydrolysis bed;
c. contacting, in a second vessel, an effluent of the first vessel with:
   i. a second sulfur guard bed containing material effective to remove and/or react sulfur-containing compounds,
   ii. a deoxygenation catalyst bed located downstream of the sulfur guard bed;
d. wherein the fermentable gas stream comprises less than 100 ppm oxygen, less than 1 ppm acetylene, and less than 1 ppm hydrogen cyanide.

The process may further comprise contacting, in the vessel, the heated input gas stream with a hydrocarbon removal adsorbent bed located upstream of the hydrolysis catalyst bed or contacting the input gas stream, in a module upstream of the heating and the vessel, with a hydrocarbon removal adsorbent bed.

The hydrolysis catalyst bed and the first sulfur guard bed may be a physical mixture forming a combined hydrolysis and sulfur guard bed.

The process may further comprise passing at least a portion of the fermentable gas stream to a bioreactor, wherein the bioreactor contains a culture comprising a fermentation broth and at least one C1-fixing microorganism.

At least a portion of the input gas stream may be a synthesis gas and/or a producer gas.

The process may further comprise measuring the concentration of impurities in the input gas stream and/or the fermentable gas stream.

The input gas stream may comprise CO, CO₂, H₂, or any combination thereof.

The first and or second sulfur guard bed material may be zinc oxide or copper and zinc oxide supported on alumina.

The deoxygenation catalyst may comprise copper supported on alumina, silica, titania, ceria, lanthana, carbon, silica-alumina, or a zeolite.

The hydrocarbon removal adsorbent may be activated carbon.

The hydrolysis bed and the first sulfur guard bed may be a combined hydrolysis and first sulfur guard bed comprising bifunctional zinc oxide on alumina support or copper and zinc oxide on alumina support.

The disclosure further provides an apparatus comprising:
a. a heating device having a heating device gas inlet and a heating device gas outlet;
b. a first vessel having a first vessel gas inlet in fluid communication with the heating device gas outlet and a first vessel gas outlet wherein the first vessel contains at least two beds comprising:
   i. a hydrolysis catalyst bed comprising alumina;
   ii. a first sulfur guard bed comprising zinc oxide or copper and zinc oxide on an alumina support located concurrent with or downstream of the hydrolysis bed;
c. a second vessel having a second vessel gas inlet in fluid communication with the first vessel gas outlet and a second vessel fermentable gas outlet wherein the second vessel contains at least two beds comprising:
   i. a second sulfur guard bed comprising zinc oxide or copper and zinc oxide on an alumina support;
   ii. a deoxygenation catalyst bed located downstream of the second sulfur guard bed, wherein the deoxygenation catalyst comprises copper supported on alumina, silica, titania, ceria, lanthana, carbon, silica-alumina, or zeolites; and
d. a bioreactor having a bioreactor gas inlet in fluid communication with the second vessel fermentable gas outlet and a bioreactor fermentation broth output wherein the bioreactor comprises at least one C1-fixing microorganism.

The apparatus may further comprise at least one monitoring device in communication with the vessel gas inlet, the vessel fermentable gas outlet, or both.

The apparatus may further comprise a hydrocarbon removal bed comprising activated charcoal located in the vessel upstream of the hydrolysis catalyst bed.

The apparatus may further comprise a hydrocarbon removal module comprising activated charcoal and having a hydrocarbon removal module gas inlet and hydrocarbon removal module gas outlet, the hydrocarbon removal module gas outlet in fluid communication with the heating device gas inlet.

### BRIEF DESCRIPTION OF THE DRAWINGS

The following detailed description is merely exemplary in nature and is not intended to limit the various embodiments or the application and uses thereof. Furthermore, there is no intention to be bound by any theory presented in the preceding background or the following detailed description. The Figures have been simplified by the deletion of a large number of apparatuses customarily employed in a process of this nature which are not specifically required to illustrate the performance of the invention. Furthermore, the illustration of the process of this disclosure in the embodiment of a specific drawing is not intended to limit the invention to specific embodiments. Some embodiments may be described by reference to the process configuration shown in the figures, which relate to both apparatus and methods to carry out the disclosure. Any reference to method includes reference to an apparatus unit or equipment that is suitable to carry out the step, and vice versa.

FIG. 1 shows a process flow scheme depicting the location of a hydrocarbon removal bed, a hydrolysis bed, a sulfur guard bed, and a deoxygenation bed all located within a common vessel in accordance with one embodiment of the disclosure.

FIG. 2 shows a process flow scheme depicting the location of a hydrolysis bed, a sulfur guard bed, and a deoxygenation bed all located within a common vessel in accordance with one embodiment of the disclosure.

FIG. 3 shows a process flow scheme depicting the location of a hydrocarbon removal bed, a hydrolysis bed, and a first sulfur guard bed, all located within a first common vessel, and a second sulfur guard bed and a deoxygenation bed both located within a second common vessel in accordance with one embodiment of the disclosure.

FIG. 4 shows a process flow scheme depicting the location of a hydrolysis bed, and a first sulfur guard bed, both located within a first common vessel, and a second sulfur guard bed and a deoxygenation bed both located within a second common vessel in accordance with one embodiment of the disclosure.

### DETAILED DESCRIPTION

The present disclosure provides systems and methods for improving carbon capture and improving overall production yield in refining and chemical manufacturing facilities by integrating into existing oil and gas infrastructure microbial fermentation that converts carbon sources that would otherwise be vented to the atmosphere or discarded to one or more products. More specifically, the present disclosure relates to systems and methods for incorporating gas fermentation into oil and gas production and refining and chemical complexes to convert various feedstock, waste gas, and other gas by-products into useful products, such as ethylene, ethanol, and the like.

The present disclosure provides systems and methods for improving carbon capture and utilization by integrating a series of impurity removal/reaction beds wherein impurities may be removed from and input gas, where the impurities may inhibit the downstream removal beds and/or a downstream gas fermentation process. A selection of adsorbents and catalysts are identified, and an order in which to position the adsorbents and catalysts is provided. Two or more of the adsorbents and or catalysts may be co-located as beds within a single vessel.

### Definitions

It is to be understood that the terminology used herein is for the purpose of describing particular embodiments only and is not intended to be limiting. Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

Also as used herein, “and/or” refers to and encompasses any and all possible combinations of one or more of the associated listed items, as well as the lack of combinations when interpreted in the alternative (“or”).

The term “substrate comprising carbon monoxide” and like terms should be understood to include any substrate in which carbon monoxide is available to one or more strains of bacteria for growth and/or fermentation, for example.

The term “gaseous substrates comprising carbon monoxide” includes any gas which contains carbon monoxide. The gaseous substrate may contain a significant proportion of CO, such as at least from about 5% to about 100% CO by volume.

The term “C1 carbon” and like terms should be understood to refer to carbon sources that are suitable for use by a microorganism, particularly those of the gas fermentation process disclosed herein. C1 carbon may include, but should not be limited to, carbon monoxide (CO), carbon dioxide (CO₂), and methane (CH₄), methanol (CH₃OH), and formate (HCOOH).

The term “substrate comprising carbon dioxide” and like terms should be understood to include any substrate in which carbon dioxide is available to one or more strains of bacteria for growth and/or fermentation, for example.

The term “gaseous substrates comprising carbon dioxide” includes any gas which contains carbon dioxide. The gaseous substrate may contain a significant proportion of CO₂, such as from at least about 5% to about 100% CO₂ by volume.

The term “co-substrate” refers to a substance that, while not necessarily being the primary energy and material source for product synthesis, can be utilized for product synthesis when added to another substrate, such as the primary substrate.

The term “directly”, as used in relation to the passing of industrial off or gases to a bioreactor, is used to mean that no or minimal processing or treatment steps, such as cooling and particulate removal are performed on the gases prior to them entering the bioreactor (note: an oxygen removal step may be required for anaerobic fermentation).

The terms “fermenting,” “fermentation process,” “fermentation reaction,” and like terms as used herein, are intended to encompass both the growth phase and product biosynthesis phase of the process. As is described further herein, in some embodiments the bioreactor may comprise a primary bioreactor and a secondary bioreactor.

The term “nutrient medium” as used herein should be understood as the solution added to the fermentation broth containing nutrients and other components appropriate for the growth of the microorganism culture.

The terms “primary bioreactor” or “first reactor” as used herein this term is intended to encompass one or more reactors that may be connected in series or parallel with a secondary bioreactor. The primary bioreactors generally use anaerobic or aerobic fermentation to produce a product (e.g., ethylene, ethanol, acetate, etc.) from a gaseous substrate.

The terms “secondary bioreactor” or “second reactor” as used herein are intended to encompass any number of further bioreactors that may be connected in series or in parallel with the primary bioreactors. Any one or more of these further bioreactors may also be connected to a further separator.

The term “stream” is used to refer to a flow of material into, through and away from one or more stages of a process, for example, the material that is fed to a bioreactor. The composition of the stream may vary as it passes through particular stages. For example, as a stream passes through the bioreactor.

The terms “feedstock” when used in the context of the stream flowing into a gas fermentation bioreactor (i.e., gas fermenter) or “gas fermentation feedstock” should be understood to encompass any material (solid, liquid, or gas) or stream that can provide a substrate and/or C1-carbon source to a gas fermenter or bioreactor either directly or after processing of the feedstock.

The term “waste gas” or “waste gas stream” may be used to refer to any gas stream that is either emitted directly, flared with no additional value capture, or combusted for energy recovery purposes.

The terms “synthesis gas” or “syngas” refers to a gaseous mixture that contains at least one carbon source, such as carbon monoxide (CO), carbon dioxide (CO₂), or any combination thereof, and, optionally, hydrogen (H₂) that can used as a feedstock for the disclosed gas fermentation processes and can be produced from a wide range of carbonaceous material, both solid and liquid.

The term “gasification” and the like should be interpreted as the process that converts organic or fossil fuel-based carbonaceous materials into carbon monoxide (CO), hydrogen (H₂), and carbon dioxide (CO₂).

The term “producer gas” should be interpreted to mean a gas stream typically used as an energy source for generating heat and/or power.

The terms “increasing the efficiency”, “increased efficiency” and the like, when used in relation to a fermentation process, include, but are not limited to, increasing one or more of the rate of growth of microorganisms catalysing the fermentation, the growth and/or product production rate at elevated product concentrations, the volume of desired product produced per volume of substrate consumed, the rate of production or level of production of the desired product, and the relative proportion of the desired product produced compared with other by-products of the fermentation.

“Gaseous substrates comprising carbon monoxide”, “gas stream comprising carbon monoxide” and the like, when used in herein include any gas which contains carbon monoxide.

The gas stream may contain a significant proportion of CO, such as from at least about 5% to about 100% CO by volume.

While it is not necessary for the substrate to contain any hydrogen, the presence of H₂ should not be detrimental to product and/or by-product formation in accordance with methods of the invention. In embodiments, the presence of hydrogen results in improved overall efficiency of alcohol production. For example, in embodiments, the gas stream may comprise an approx. 2:1, or 1:1, or 1:2 molar ratio of H₂:CO. In one embodiment, the gas stream comprises about 30% or less H₂ by volume, 20% or less H₂ by volume, about 15% or less H₂ by volume or about 10% or less H₂ by volume. In other embodiments, the gas stream comprises low concentrations of H₂, for example, less than 5% by volume, or less than 4% by volume, or less than 3% by volume, or less than 2% by volume, or less than 1% by volume, or is substantially hydrogen free. The gas stream may also contain some CO₂ for example, such as about 1% to about 80% CO₂ by volume, or 1% to about 30% CO₂ by volume. In one embodiment, the gas stream comprises less than or equal to about 20% CO₂ by volume. In embodiments, the gas stream comprises less than or equal to about 15% CO₂ by volume, less than or equal to about 10% CO₂ by volume, less than or equal to about 5% CO₂ by volume or substantially no CO₂.

“Gas stream” refers to any stream of substrate which is capable of being passed, for example, from one bed to another, from one bed to a bioreactor, and/or from one bed to a carbon capture means.

“Reactants” as used herein refer to a substance that takes part in and undergoes change during a chemical reaction. In embodiments, the reactants include, but are not limited to, CO, CO₂, and/or H₂.

“Microorganism inhibitors” as used herein refer to one or more impurity that slows down or prevents a particular chemical reaction or other process including microbial fermentation. In embodiments, the microorganism inhibitors include, but are not limited to, oxygen (O₂), hydrogen cyanide (HCN), acetylene (C₂H₂), and BTEX (benzene, toluene, ethyl benzene, xylene).

“Catalyst inhibitor”, “adsorbent inhibitor”, and the like, as used herein, refer to one or more substance that decreases the rate of, or prevents, a desired chemical reaction. In embodiments, the catalyst and/or adsorbent inhibitors may include but are not limited to, hydrogen sulfide (H₂S) and carbonyl sulfide (COS).

“Removal bed”, “clean-up bed”, “processing bed” and the like, includes technologies that are capable of either converting and/or removing microorganism inhibitors and/or catalyst inhibitors from the gas stream.

The term “impurities”, “contaminants”, and the like, as used herein, refers to the reactants, microorganism inhibitors, and/or catalyst inhibitors that may be found in the gas stream.

The term “treated gas” refers to the gas stream that has been passed through at least one removal bed and has had one or more impurity removed and/or converted.

The term “predetermined level”, “predetermined level of impurities”, and the like, as used herein, refer to the amount of one or more impurity deemed to be acceptable in the gas stream. The predetermined levels stated herein were identified by performing microbial tolerance experiments.

The term “fermentable gaseous substrate”, “fermentable gas stream” and the like, as used herein, refers to a gas stream that contains a predetermined level of impurities, and is capable of being used as a carbon source by C1-fixing microorganisms.

The term “carbon capture” as used herein refers to the utilization or sequestration of carbon compounds including CO₂ and/or CO from a stream comprising CO₂ and/or CO and either:
converting the CO₂ and/or CO into products; or
converting the CO₂ and/or CO into substances suitable for long term storage; or
trapping the CO₂ and/or CO in substances suitable for long term storage;
or a combination of these processes.

The term “bioreactor” includes a fermentation device consisting of one or more vessels and/or towers or piping arrangements, which includes the continuous stirred tank reactor (CSTR), immobilized cell reactor (ICR), trickle bed reactor (TBR), bubble column, gas lift fermenter, static mixer, a circulated loop reactor, a membrane reactor, such as a hollow fibre membrane bioreactor (HFM BR) or other vessel or device suitable for gas-liquid contact. The reactor may be adapted to receive a fermentable gas stream comprising CO or CO₂ or H₂ or mixtures thereof. The reactor may comprise multiple reactors (stages), either in parallel or in series. For example, the reactor may comprise a first growth reactor in which the bacteria are cultured and a second fermentation reactor, to which fermentation broth from the growth reactor may be fed and in which most of the fermentation products may be produced.

“Nutrient media” or “Nutrient medium” is used to describe bacterial growth media. Generally, this term refers to a media containing nutrients and other components appropriate for the growth of a microbial culture. The term “nutrient” includes any substance that may be utilized in a metabolic pathway of a microorganism. Exemplary nutrients include potassium, B vitamins, trace metals, and amino acids.

The term “fermentation broth” or “broth” is intended to encompass the mixture of components including nutrient media and a culture or one or more microorganisms. It should be noted that the term microorganism and the term bacteria are used interchangeably throughout the document.

The term “acid” as used herein includes both carboxylic acids and the associated carboxylate anion, such as the mixture of free acetic acid and acetate present in a fermentation broth as described herein. The ratio of molecular acid to carboxylate in the fermentation broth is dependent upon the pH of the system. In addition, the term “acetate” includes both acetate salt alone and a mixture of molecular or free acetic acid and acetate salt, such as the mixture of acetate salt and free acetic acid present in a fermentation broth as described herein.

The term “acid gas” as used herein is a classification of gas which contains mixtures of impurities in quantities making the gas acidic. Acid gas may contain large proportions of hydrogen sulfide (H₂S) and/or carbon dioxide (CO₂). Additionally, the acid gas may contain proportions of carbonyl sulfide (COS), hydrogen chloride (HCl), hydrogen fluoride (HF), and/or hydrogen cyanide (HCN).

The term “desired composition” is used to refer to the desired level and types of components in a substance, such as, for example, of a gas stream. More particularly, a gas is considered to have a “desired composition” if it contains a particular component (i.e. CO and/or CO₂) and/or contains a particular component at a particular level and/or does not contain a particular component (i.e. a contaminant harmful to the microorganisms) and/or does not contain a particular component at a particular level. More than one component may be considered when determining whether a gas stream has a desired composition. The gas stream that may be sent to the bioreactor is fermentable, such that it has a desired composition.

Unless the context requires otherwise, the phrases “fermenting”, “fermentation process” or “fermentation reaction” and the like, as used herein, are intended to encompass both the growth phase and product biosynthesis phase of the gaseous substrate.

A “microorganism” is a microscopic organism, especially a bacterium, archea, virus, or fungus. The microorganism of the invention is typically a bacterium. As used herein, recitation of “microorganism” should be taken to encompass “bacterium.”

A “parental microorganism” is a microorganism used to generate a microorganism of the invention. The parental microorganism may be a naturally occurring microorganism (i.e., a wild-type microorganism) or a microorganism that has been previously modified (i.e., a mutant or recombinant microorganism). The microorganism of the invention may be modified to express or overexpress one or more enzymes that were not expressed or overexpressed in the parental microorganism. Similarly, the microorganism of the invention may be modified to contain one or more genes that were not contained by the parental microorganism. The microorganism of the invention may also be modified to not express or to express lower amounts of one or more enzymes that were expressed in the parental microorganism. In one embodiment, the parental microorganism is Clostridium autoethanogenum, Clostridium ljungdahlii, or Clostridium ragsdalei. The parental microorganism may be Clostridium autoethanogenum LZ1561, which was deposited on Jun. 7, 2010, with Deutsche Sammlung von Mikroorganismen und Zellkulturen GmbH (DSMZ) located at Inhoffenstraße 7B, D-38124 Braunschweig, Germany on Jun. 7, 2010, under the terms of the Budapest Treaty and accorded accession number DSM23693. This strain is described in International Patent Application No. PCT/NZ2011/000144, which published as WO 2012/015317.

The term “derived from” indicates that a nucleic acid, protein, or microorganism is modified or adapted from a different (i.e., a parental or wild type) nucleic acid, protein, or microorganism, so as to produce a new nucleic acid, protein, or microorganism. Such modifications or adaptations typically include insertion, deletion, mutation, or substitution of nucleic acids or genes. Generally, the microorganism of the invention is derived from a parental microorganism. In one embodiment, the microorganism of the invention is derived from Clostridium autoethanogenum, Clostridium ljungdahlii, or Clostridium ragsdalei. In an embodiment, the microorganism of the invention is derived from Clostridium autoethanogenum LZ1561, which is deposited under DSMZ accession number DSM23693.

“Wood-Ljungdahl” refers to the Wood-Ljungdahl pathway of carbon fixation as described, i.e., by Ragsdale, Biochim Biophys Acta, 1784: 1873-1898, 2008. “Wood-Ljungdahl microorganisms” refers, predictably, to microorganisms containing the Wood-Ljungdahl pathway. Generally, the microorganism of the invention contains a native Wood-Ljungdahl pathway. Herein, a Wood-Ljungdahl pathway may be a native, unmodified Wood-Ljungdahl pathway or it may be a Wood-Ljungdahl pathway with some degree of genetic modification (i.e., overexpression, heterologous expression, knockout, etc.) so long as it still functions to convert CO, CO₂, and/or H₂ to acetyl-CoA.

“C1” refers to a one-carbon molecule, for example, CO, CO₂, CH₄, or CH₃OH. “C1-oxygenate” refers to a one-carbon molecule that also comprises at least one oxygen atom, for example, CO, CO₂, or CH₃OH. “C1-carbon source” refers a one carbon-molecule that serves as a partial or sole carbon source for the microorganism of the invention. For example, a C1-carbon source may comprise one or more of CO, CO₂, CH₄, CH₃OH, or CH₂O₂. The C1-carbon source may comprise one or both of CO and CO₂. A “C1-fixing microorganism” is a microorganism that has the ability to produce one or more products from a C1-carbon source. Typically, the microorganism of the invention is a C1-fixing bacterium.

An “anaerobe” is a microorganism that does not require oxygen for growth. An anaerobe may react negatively or even die if oxygen is present above a certain threshold. However, some anaerobes are capable of tolerating low levels of oxygen (i.e., 0.000001-5% oxygen). Typically, the microorganism of the invention is an anaerobe.

“Acetogens” are obligately anaerobic bacteria that use the Wood-Ljungdahl pathway as their main mechanism for energy conservation and for the synthesis of acetyl-CoA and acetyl-CoA-derived products, such as acetate (Ragsdale, Biochim Biophys Acta, 1784: 1873-1898, 2008). In particular, acetogens use the Wood-Ljungdahl pathway as a (1) mechanism for the reductive synthesis of acetyl-CoA from CO₂, (2) terminal electron-accepting, energy conserving process, (3) mechanism for the fixation (assimilation) of CO₂ in the synthesis of cell carbon (Drake, Acetogenic Prokaryotes, In: The Prokaryotes, 3^{rd} edition, p. 354, New York, NY, 2006). All naturally occurring acetogens are C1-fixing, anaerobic, autotrophic, and non-methanotrophic. Typically, the microorganism of the invention is an acetogen.

An “ethanologen” is a microorganism that produces or is capable of producing ethanol. Typically, the microorganism of the invention is an ethanologen.

An “autotroph” is a microorganism capable of growing in the absence of organic carbon. Instead, autotrophs use inorganic carbon sources, such as CO and/or CO₂. Typically, the microorganism of the invention is an autotroph.

A “carboxydotroph” is a microorganism capable of utilizing CO as a sole source of carbon and energy. Typically, the microorganism of the invention is a carboxydotroph.

A “methanotroph” is a microorganism capable of utilizing methane as a sole source of carbon and energy. In certain embodiments, the microorganism of the invention is a methanotroph or is derived from a methanotroph. In other embodiments, the microorganism of the invention is not a methanotroph or is not derived from a methanotroph.

“Substrate” refers to a carbon and/or energy source for the microorganism of the invention. Typically, the substrate is gaseous and comprises a C1-carbon source, for example, CO, CO₂, and/or CH₄. The substrate may comprise a C1-carbon source of CO or CO+CO₂. The substrate may further comprise other non-carbon components, such as H₂, N₂, or electrons.

The term “co-substrate” refers to a substance that, while not necessarily being the primary energy and material source for product synthesis, can be utilized for product synthesis when added to another substrate, such as the primary substrate.

The substrate and/or C1-carbon source may be a gas obtained as a by-product of an industrial process or from another source, such as combustion engine exhaust fumes, biogas, landfill gas, direct air capture, flaring, or from electrolysis. The substrate and/or C1-carbon source may be syngas generated by pyrolysis, torrefaction, or gasification. In other words, carbon in solid or liquid materials may be recycled by pyrolysis, torrefaction, or gasification to generate syngas which is used as the substrate and/or C1-carbon source in gas fermentation. The substrate and/or C1-carbon source may be natural gas. The substrate and/or C1-carbon source carbon dioxide from conventional and unconventional gas production. The substrate and/or C1-carbon source may be a gas comprising methane. Gas fermentation processes are flexible and any of these substrate and/or C1-carbon sources may be employed.

In certain embodiments, the industrial process source of the substrate and/or C1 carbon source is selected from ferrous metal products manufacturing, such as a steel manufacturing, non-ferrous products manufacturing, petroleum refining, electric power production, carbon black production, paper and pulp manufacturing, ammonia production, methanol production, coke manufacturing, petrochemical production, carbohydrate fermentation, cement making, aerobic digestion, anaerobic digestion, catalytic processes, natural gas extraction, cellulosic fermentation, oil extraction, industrial processing of geological reservoirs, processing fossil resources such as natural gas coal and oil, landfill operations, or any combination thereof. Examples of specific processing steps within an industrial process include catalyst regeneration, fluid catalyst cracking, and catalyst regeneration. Air separation and direct air capture are other suitable industrial processes. Specific examples in steel and ferroalloy manufacturing include blast furnace gas, basic oxygen furnace gas, coke oven gas, direct reduction of iron furnace top-gas, and residual gas from smelting iron. Other general examples include flue gas from fired boilers and fired heaters, such as natural gas, oil, or coal fired boilers or heaters, and gas turbine exhaust. Another example is the flaring of compounds such as at oil and gas production sites. In these embodiments, the substrate and/or C1-carbon source may be captured from the industrial process before it is emitted into the atmosphere, using any known method.

The substrate and/or C1-carbon source may be synthesis gas known as syngas, which may be obtained from reforming, partial oxidation, plasma, or gasification processes. Examples of gasification processes include gasification of coal, gasification of refinery residues, gasification of petroleum coke, gasification of biomass, gasification of lignocellulosic material, gasification of wood, gasification of black liquor, gasification of municipal solid waste, gasification of municipal liquid waste, gasification of industrial solid waste, gasification of industrial liquid waste, gasification of refuse derived fuel, gasification of sewerage, gasification of sewerage sludge, gasification of sludge from wastewater treatment, gasification of landfill gas, gasification of biogas such as when biogas is added to enhance gasification of another material. Examples of reforming processes include, steam methane reforming, steam naphtha reforming, reforming of natural gas, reforming of biogas, reforming of landfill gas, reforming of coke oven gas, reforming of pyrolysis off-gas, reforming of ethylene production off-gas, naphtha reforming, and dry methane reforming. Examples of partial oxidation processes include thermal and catalytic partial oxidation processes, catalytic partial oxidation of natural gas, partial oxidation of hydrocarbons, partial oxidation of biogas, partial oxidation of landfill gas, or partial oxidation of pyrolysis off-gas. Examples of municipal solid waste include tires, plastics, refuse derived fuel, and fibers such as in shoes, apparel, and textiles. Municipal solid waste may be simply landfill-type waste and may be sorted or unsorted. Examples of biomass may include lignocellulosic material and microbial biomass. Lignocellulosic material may include agriculture by-products, forest by-products, and some industrial by-products.

Biomass may be created as by-products of “nature-based solutions” (NbS) and thus natured-based solutions may provide feedstock to the gas fermentation process. Nature-based solutions is articulated by the European Commission as solutions inspired and supported by nature, which are cost-effective, simultaneously provide environmental, social, and economic benefits and help build resilience. Such solutions bring more, and more diverse, nature and natural features and processes into cities, landscapes, and seascapes, through locally adapted, resource-efficient, and systemic interventions. Nature-based solutions must benefit biodiversity and support the delivery of a range of ecosystem services. Through the use of NbS healthy, resilient, and diverse ecosystems (whether natural, managed, or newly created) can provide solutions for the benefit of both societies and overall biodiversity. Examples of nature-based solutions include natural climate solutions (conservation, restoration and improved land management that increase carbon storage or avoid greenhouse gas emissions in landscapes and wetlands across the globe), halting biodiversity loss, socio-economic impact efforts, habitat restoration, and health and wellness efforts with respect to air and water. Biomass produced through nature-based solutions may be used as feedstock to gas fermentation processes.

As shown, the optional step of a gasification process in the overall gas fermentation process greatly increases suitable feedstocks to the overall gas fermentation process as compared to gaseous feedstocks alone. Further, incentives achieved may extend beyond items such as carbon credits, and into the natural based solutions space.

The substrate and/or C1-carbon source may be a gas stream comprising methane. Such a methane containing gas may be obtained from: fossil methane emissions such as during fracking, wastewater treatment, livestock, agriculture, and municipal solid waste landfills. It is also envisioned that the methane may be burned to produce electricity or heat and the C1 by-products may be used as the substrate or carbon source. The substrate and/or C1-carbon source may be a gas stream comprising natural gas.

The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, microbial inhibitors, or dust particles, and/or increase the concentration of desirable components.

In certain embodiments, the fermentation is performed in the absence of carbohydrate substrates, such as sugar, starch, lignin, cellulose, or hemicellulose.

The microorganism contained in the bioreactor may be cultured with the feedstock and produce one or more gas fermentation products. For instance, the microorganism may produce or may be engineered to produce ethanol (WO 2007/117157, U.S. Pat. No. 7,972,824), acetate (WO 2007/117157, U.S. Pat. No. 7,972,824), 1-butanol (WO 2008/115080, U.S. Pat. No. 8,293,509, WO 2012/053905, U.S. Pat. No. 9,359,611 and WO 2017/066498, U.S. Pat. No. 9,738,875), butyrate (WO 2008/115080, U.S. Pat. No. 8,293,509), 2,3-butanediol (WO 2009/151342, U.S. Pat. No. 8,658,408 and WO 2016/094334, U.S. Pat. No. 10,590,406), lactate (WO 2011/112103, U.S. Pat. No. 8,900,836), butene (WO 2012/024522, US2012/045807), butadiene (WO 2012/024522, US 2012/045807), methyl ethyl ketone (2-butanone) (WO 2012/024522, US 2012/045807 and WO 2013/185123, U.S. Pat. No. 9,890,384), ethanol which is then converted to ethylene (WO 2012/026833, US 2013/157,322), acetone (WO 2012/115527, U.S. Pat. No. 9,410,130), isopropanol (WO 2012/115527 U.S. Pat. No. 9,410,130), lipids (WO 2013/036147 U.S. Pat. No. 9,068,202), 3-hydroxypropionate (3-HP) (WO 2013/180581, U.S. Pat. No. 9,994,878), terpenes, including isoprene (WO 2013/180584, U.S. Pat. No. 10,913,958), fatty acids (WO 2013/191567 U.S. Pat. No. 9,347,076), 2-butanol (WO 2013/185123 U.S. Pat. No. 9,890,384), 1,2-propanediol (WO 2014/036152, U.S. Pat. No. 9,284,564), 1-propanol (WO 2014/0369152, U.S. Pat. No. 9,284,564), 1 hexanol (WO 2017/066498, U.S. Pat. No. 9,738,875), 1 octanol (WO 2017/066498, U.S. Pat. No. 9,738,875), chorismate-derived products (WO 2016/191625, U.S. Pat. No. 10,174,303), 3-hydroxybutyrate (WO 2017/066498, U.S. Pat. No. 9,738,875), 1,3-butanediol (WO 2017/066498, U.S. Pat. No. 9,738,875), 2-hydroxyisobutyrate or 2-hydroxyisobutyric acid (WO 2017/066498, U.S. Pat. No. 9,738,875), isobutylene (WO 2017/066498, U.S. Pat. No. 9,738,875), adipic acid (WO 2017/066498, U.S. Pat. No. 9,738,875), 1,3-hexanediol (WO 2017/066498, U.S. Pat. No. 9,738,875), 3-methyl-2-butanol (WO 2017/066498, U.S. Pat. No. 9,738,875), 2-buten-1-ol (WO 2017/066498, U.S. Pat. No. 9,738,875), isovalerate (WO 2017/066498, U.S. Pat. No. 9,738,875), isoamyl alcohol (WO 2017/066498, U.S. Pat. No. 9,738,875), and/or monoethylene glycol (WO 2019/126400, U.S. Pat. No. 11,555,209) in addition to 2-phenylethanol (WO 2021/188190, US 2021/0292732). In certain embodiments, microbial biomass itself may be considered a product. One or more of these products may be further converted to produce at least one component of diesel, jet fuel, and/or gasoline.

A “native product” is a product produced by a genetically unmodified microorganism. For example, ethanol, acetate, and 2,3-butanediol are native products of Clostridium autoethanogenum, Clostridium ljungdahlii, and Clostridium ragsdalei. A “non-native product” is a product that is produced by a genetically modified microorganism but is not produced by a genetically unmodified microorganism from which the genetically modified microorganism is derived.

“Selectivity” refers to the ratio of the production of a target product to the production of all fermentation products produced by a microorganism. The microorganism of the invention may be engineered to produce products at a certain selectivity or at a minimum selectivity. In one embodiment, a target product accounts for at least about 5% by mass, 10% by mass, 15% by mass, 20% by mass, 30% by mass, 50% by mass, or 75% by mass of all fermentation products produced by the microorganism of the invention. In one embodiment, the target product accounts for at least 10% by mass of all fermentation products produced by the microorganism of the invention, such that the microorganism of the invention has a selectivity for the target product of at least 10% by mass. In another embodiment, the target product accounts for at least 30% by mass of all fermentation products produced by the microorganism of the invention, such that the microorganism of the invention has a selectivity for the target product of at least 30% by mass.

Typically, the culture is performed in a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or two or more such reactors. As used herein, the terms “culture” and “fermentation” are used interchangeably. These terms encompass both the growth phase and the product biosynthesis phase of the culture/fermentation process.

The culture is generally maintained in an aqueous culture medium that contains nutrients, vitamins, and/or minerals sufficient to permit growth of the microorganism. The aqueous culture medium may be an anaerobic microbial growth medium, such as a minimal anaerobic microbial growth medium. Suitable media are well known in the art.

The culture/fermentation should desirably be carried out under appropriate conditions to produce the target product. Typically, the culture/fermentation is performed under anaerobic conditions. Reaction conditions to consider include pressure (or partial pressure), temperature, gas flow rate, liquid flow rate, media pH, media redox potential, agitation rate (if using a continuous stirred tank reactor), inoculum level, maximum gas substrate concentrations to ensure that gas in the liquid phase does not become limiting, and maximum product concentrations to avoid product inhibition. In particular, the rate of introduction of the substrate may be controlled to ensure that the concentration of gas in the liquid phase does not become limiting, since products may be consumed by the culture under gas-limited conditions.

Operating a bioreactor at elevated pressures allows for an increased rate of gas mass transfer from the gas phase to the liquid phase. Accordingly, the culture/fermentation may be performed at pressures higher than atmospheric pressure. Also, since a given gas conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. This, in turn, means that the retention time, defined as the liquid volume in the bioreactor divided by the input gas flow rate, can be reduced when bioreactors are maintained at elevated pressure rather than atmospheric pressure. The optimum reaction conditions will depend partly on the microorganism used. However, in general, the fermentation may be operated at a pressure higher than atmospheric pressure. Also, since a given gas conversion rate is in part a function of substrate retention time and achieving a desired retention time, in turn, dictates the required volume of a bioreactor, the use of pressurized systems can greatly reduce the volume of the bioreactor required, and consequently the capital cost of the fermentation equipment.

Target products may be separated or purified from a fermentation broth using any method or combination of methods known in the art, including, for example, fractional distillation, vacuum distillation, evaporation, pervaporation, gas stripping, phase separation, and extractive fermentation, including, for example, liquid-liquid extraction. In certain embodiments, target products are recovered from the fermentation broth by continuously removing a portion of the broth from the bioreactor, separating microbial cells from the broth (conveniently by filtration), and recovering one or more target products from the broth. Alcohols and/or acetone may be recovered, for example, by distillation. Acids may be recovered, for example, by adsorption on activated charcoal. Separated microbial cells may be returned to the bioreactor. The cell-free permeate remaining after target products have been removed may be also returned to the bioreactor. Additional nutrients (such as B vitamins) may be added to the cell-free permeate to replenish the medium before it is returned to the bioreactor.

### Description

Particularly in a revamp situation where the number of vessels are limited, incorporating various beds together in a vessel all operating under common operating parameters may be successfully accomplished. Various gas impurities may be converted and/or removed from the gas stream, in a stepwise manner, and where impurities may be harmful to downstream beds they are removed and/or converted upstream from those beds, which then allows for subsequent removal of other gas impurities, and later feeding of a fermentable gas stream to a bioreactor where the gas may be processed by gas fermenting microorganisms to create useful products. The conversion and/or removal of these impurities is achieved without consuming desired compounds and without creating other undesired compounds. In embodiments, the fermentable gas stream may be passed to a carbon capture means for storage.

In embodiments, the input gas stream is passed, in series, to the following beds for processing: (1) optional hydrocarbon removal (2) hydrolysis; (3) optional sulfur compound removal; (4) optional catalytic hydrogenation; and (5) deoxygenation. Each bed is utilized to remove and/or convert one or more impurity in the gas stream. As many deoxygenation catalysts are sulfur sensitive, the sulfur removal bed may be advantageous and thus included and located upstream of the deoxygenation catalyst bed. Two or more of the beds may be located within a single vessel. Depending upon vessel availability, two, three, four, or all 5 beds may be located in a single vessel. Co-location within a single vessel is often called stacked bed configuration.

The challenge in co-locating two or more beds of different material in the same vessel is that all beds will be subjected to the same operating conditions. The specific materials selected must remain unaffected by treatment of one of the beds in the series. As an example, a particular deoxygenation catalyst operates best in a reduced form. The rest of the beds co-located in the same vessel as the deoxygenation catalyst must remain substantially unaffected by the reduction temperature and the reduction gas needed to reduce the deoxygenation catalyst.

### Hydrocarbon Removal Bed

In an embodiment, a hydrocarbon removal bed may be employed to removes hydrocarbons such as BTEX. A suitable adsorbent for the hydrocarbon removal bed may be silicas, aluminas, silica-aluminas, activated carbon or any combination thereof. The input gas is contacted with the adsorbent and one or more hydrocarbons are adsorbent and therefore removed from the gas stream. The adsorbent may be in the form of a fixed bed, and the bed may be operated in a static mode, a temperature swing adsorption mode, or in a pressure swing adsorption mode.

In some embodiments the hydrocarbon removal bed may be Pressure Swing Adsorption (PSA) which is an adiabatic process used for the purification of gases to remove accompanying impurities by adsorption through suitable adsorbents in fixed beds contained in vessels under high pressure. Regeneration of adsorbents is accomplished by counter current depressurization and by purging at low pressure with previously recovered treated gas. To obtain a continuous flow of product, at least two adsorbers may be provided such that at least one adsorber is receiving, treating, and sending a treated gas stream to further treatment beds, and at least one adsorber is used to perform the regeneration of the one or more adsorbers that send the treated gas stream to further treatment beds. Common adsorbents may readily be selected by one of skill in the art dependent on the type of impurity to be absorbed and removed. Suitable adsorbents include zeolitic molecular sieves, activated carbon, silica gel or activated alumina. Combinations of absorbent beds may be used on top of one another, thereby dividing the adsorber contents into a number of distinct zones. PSA involves a pendulating swing in parameters such as pressure, temperature, flow, and composition of gaseous and adsorbed phase. Purification or separation of gases using PSA normally takes place at near ambient feed gas temperatures, whereby the components to be removed are selectively adsorbed. Adsorption should ideally be sufficiently reversible to enable regeneration of adsorbents at similar ambient temperature. Additionally, adsorption may be conducted such that the production of undesirable compounds is avoided, or at least minimized.

In some embodiments the hydrocarbon removal bed may be Temperature Swing Adsorption (TSA) which is technique used for the purification of gases to remove accompanying impurities by adsorption through suitable adsorbents in fixed beds contained in vessels under an adsorption temperature. Regeneration of adsorbent is accomplished by counter current flow under a second desorption temperature and optionally purging at desorption temperature with previously recovered treated gas. To obtain a continuous flow of product, at least two adsorbers may be provided such that at least one adsorber is receiving, treating, and sending a treated gas stream to further treatment beds, and at least one adsorber is used to perform the regeneration of the one or more adsorbers that send the treated gas stream to further treatment beds. Common adsorbents may readily be selected by one of skill in the art dependent on the type of impurity to be absorbed and removed. Suitable adsorbents include zeolitic molecular sieves, activated carbon, silica gel or activated alumina. Combinations of absorbent beds may be used on top of one another, thereby dividing the adsorber contents into a number of distinct zones. TSA involves a pendulating swing in parameters such as pressure, temperature, flow, and composition of gaseous and adsorbed phase. Adsorption should ideally be sufficiently reversible to enable regeneration of adsorbents. Additionally, adsorption may be conducted such that the production of undesirable compounds is avoided, or at least minimized.

### Hydrolysis Bed

Hydrogen cyanide (HCN) and carbonyl sulfide (COS) are two anticipated impurities that first are chemically reacted with water in advance of being successfully removed from the gas stream. In applications where a high sulfur gas stream is utilized, converting COS to hydrogen sulfide (H₂S) may be necessary because many commercial processes cannot efficiently remove sulfur in the form of COS. This conversion occurs according to the following reaction:

COS+H₂O↔H₂S+CO₂

This conversion can be achieved using any technology capable of converting COS to H₂S. In various embodiments, the hydrolysis bed utilizes a metal oxide catalyst to perform the conversion. In embodiments, an alumina catalyst is used to perform the conversion. The alumina may be any form of alumina or silica-alumina such as gamma alumina, theta alumina, delta alumina, and alpha alumina with gamma.

In embodiments, the hydrolysis step may include a multibed approach to convert COS and remove H₂S. In embodiments, the first bed utilizes a conversion bed whereby COS is converted to H₂S. An example of such a conversion bed includes the BASF SELEXSORB™ COS. In embodiments, the second bed utilizes an iron-based adsorbent, such as the high-capacity non-hazardous granular media sold under the tradename “AxTrap 4001”, which removes H₂S.

In embodiments, the gas stream is fed to the hydrolysis bed to convert and/or remove one or more impurity from the gas stream. In certain instances, the post-hydrolysis gas stream is depleted in at least one impurity selected from the group comprising: COS and/or HCN.

### Sulfur Guard Bed

A sulfur guard bed, also referred to as an acid gas removal bed, refers to a process by which hydrogen sulfide (H₂S) and/or carbon dioxide (CO₂), as well as other sulfur containing compounds or acid gases, are separated from the gas stream. The sulfur guard bed is optional, particularly in embodiments were the gas feedstock has little to no sulfur compounds. In embodiments were a sulfur guard bed is employed, the material selected for use in the sulfur guard bed will be exposed to the same temperature as the deoxygenation catalyst bed. Therefore, the materials selected for use in the sulfur guard bed are materials that do not undergo reduction at the temperature of the deoxygenation catalyst bed. In certain instances, the sulfur guard bed utilizes a zinc oxide (ZnO) catalyst to remove hydrogen sulfide (H₂S) from the gas stream. In other instances, the sulfur guard bed utilizes a copper and zinc oxide may be composited with one or more aluminas. The resulting composite adsorbent may be considered to be a bound adsorbent or a supported adsorbent. Temperatures around 600° C. are needed to reduce zinc oxide. The deoxygenation catalyst bed is operated at temperatures below 600° C. and therefore ZnO or copper and zinc oxide supported on alumina is a particularly suitable selection of adsorbent for the sulfur guard bed.

An optional carbon dioxide adsorption bed, or additional acid gas removal bed, may be used after the original sulfur guard bed. The carbon dioxide adsorption bed is used to remove carbon dioxide (CO₂) from the gas stream to bring the carbon dioxide levels within the desired range. In these embodiments, the treated gas from the sulfur guard bed may be sent to the carbon dioxide adsorption bed prior to being sent to the optional catalytic hydrogenation bed. In embodiments that bypass or do not employ the optional catalytic hydrogenation bed, the treated gas from the sulfur guard bed may be sent directly to the deoxygenation bed.

In embodiments, the gas stream is fed to the acid gas removal bed to convert and/or remove one or more impurity from the gas stream. In certain instances, the acid gas-depleted stream is depleted in at least one impurity selected from the group comprising: carbon dioxide (CO₂), hydrogen sulfide (H₂S), and hydrogen cyanide (HCN).

In an embodiment, the hydrolysis catalyst bed and the sulfur guard bed may be mixed into a physical mixture and employed as a single bed. Optionally, the hydrocarbon removal adsorbent may also be present in the physical mixture. In another embodiment the hydrolysis catalyst functionality and the sulfur guard functionality may be combined into a single composite and used as a combined function bed in the vessel. One example of the bifunctional functionality composite is zinc oxide on aluminas, silicas, silica-aluminas.

### Catalytic Hydrogenation Bed

Acetylene (C₂H₂) acts as a microorganism inhibitor. To remove acetylene a catalytic hydrogenation bed may be utilized. Catalytic hydrogenation is treatment with hydrogen in the presence of a catalyst such as, but not limited to, nickel, palladium, or platinum. There is not one universal catalyst suitable for the hydrogenation of acetylene. The choice of catalyst greatly depends upon the gas composition and operating conditions. In embodiments, palladium is used as the catalyst. In embodiments, palladium on alumina (Pd/Al₂O₃) is used as the catalyst. An example of such a catalyst is the BASF™ R 0-20/47.

Inhibitors reduce the activity of palladium. Sulfur compounds represent potential palladium inhibitors. Compounds such as hydrogen sulfide (H₂S) or carbonyl sulfide (COS) adsorb on palladium and may alter the reaction sites. In embodiments, known palladium inhibitors are removed and/or converted prior to catalytic hydrogenation.

In embodiments, a catalytic hydrogenation bed may be unnecessary for acetylene removal. In addition to being removed by a catalytic hydrogenation bed, acetylene may be removed from the gas stream by certain deoxygenation beds. In embodiments where the catalytic hydrogenation bed is unnecessary, the catalytic hydrogenation bed may be bypassed and/or not included in the process. An example of when the catalytic hydrogenation bed is unnecessary is when acetylene levels are low enough such that they can be effectively removed via the other beds. In embodiments where the acetylene levels are low enough, the gas stream may be passed from the acid gas removal bed to the deoxygenation bed, bypassing the catalytic hydrogenation bed.

In embodiments, the gas stream is fed to the catalytic hydrogenation bed to convert and/or remove one or more impurity from the gas stream. In certain instances, the post-hydrogenation stream is depleted in at least acetylene (C₂H₂).

### Deoxygenation Bed

Oxygen (O₂) may be a microorganism inhibitor. Therefore, the oxygen in the gas stream may need to be reduced to acceptable levels. To reduce the levels of oxygen in the gas stream a deoxygenation bed may be utilized. The reduction of oxygen levels may be achieved through any suitable means. In embodiments, the deoxygenation bed utilizes a catalytic process whereby oxygen (O₂) is reduced to either carbon dioxide (CO₂) or water (H₂O). In embodiments, the catalyst used in the deoxygenation bed is copper containing. The copper may be in reduced form. The copper may be compounded with other elements such as those in IUPAC Group 8, IUPAC Group 9, and/or IUPAC Group 10, such as iron (Fe), cobalt (Co), nickel (Ni) ruthenium (Ru) rhodium (Rh), platinum (Pt), palladium (Pd), osmium (Os), iridium (Ir), to form the catalyst. The copper may be in a molar ratio with another element of up to about 1:1. An example of a such a catalyst is the BASF PURISTAR™ R 3-15 or BASF CU 0226S.

In embodiments, the deoxygenation bed can be used to effectively reduce the level of acetylene in the gas stream thereby allowing for the catalytic hydrogenation step to be bypassed. One notable difference between the removal of acetylene by the catalytic hydrogenation bed and the deoxygenation bed is the production of ethane (C₂H₆). Removal of acetylene by the deoxygenation bed produces higher amounts of ethane than the removal of acetylene by the catalytic hydrogenation bed. However, due to the robust nature of the microorganism used in the gas fermentation process, the level of ethane produced by the deoxygenation bed was not harmful to the microorganism, and thus, in embodiments, the catalytic hydrogenation bed was able to be bypassed.

Another notable difference between the catalytic hydrogenation bed and the deoxygenation bed is the production of methanol (CH₃OH). Methanol may be produced when utilizing any copper-based deoxygenation bed. In instances where a copper-based deoxygenation bed is utilized to remove acetylene, the removal process produces higher amounts of methanol relative to a removal process utilizing a catalytic hydrogenation bed. However, due to the robust nature of the microorganism used in the subsequent gas fermentation process, the level of methanol produced by the deoxygenation bed was not harmful to the microorganism, and thus, in embodiments, the catalytic hydrogenation bed was able to be bypassed.

In addition to the impurities above, certain deoxygenation beds may be used to effectively reduce mercury (Hg). Not all gas streams will contain mercury (Hg). However, the treatment process is designed to effectively treat gas streams from a number of sources, some of which may contain mercury (Hg). Therefore, in certain instances where the gas stream contains mercury (Hg), a deoxygenation bed may be utilized to effectively remove mercury (Hg) from the gas stream. When mercury (Hg) is removed from the gas stream by the deoxygenation bed, the post-deoxygenation stream may be depleted in mercury (Hg).

In embodiments, the gas stream is fed to the deoxygenation bed in order to convert and/or remove one or more impurity from the gas stream. In certain instances, the post-deoxygenation stream is depleted in at least oxygen (O₂) and/or acetylene (C₂H₂). In various instances, the post-deoxygenation stream is depleted in mercury (Hg) in addition to oxygen (O₂) and/or acetylene (C₂H₂).

In some embodiments, catalysts traditionally employed for hydrotreating may be used as the deoxygenation catalyst. Such catalysts are metal catalyst such as molybdenum and tungsten promoted by nickel and cobalt. Catalyst metals may be impregnated on a support material that provides significant surface area. Support materials may be alumina, silica, magnesia, zirconia, and zeolites. These supports provide high surface area and pore size that has the ability to withstand operating pressure and temperature conditions and support the required reactions. Catalyst shapes may include cylinder, hollow, trilobe, sphere, and quad-lobe. These catalysts are activated by partially or fully sulfiding. When selecting the material to use to sulfide the catalyst, care should be taken to avoid the generation of sulfur containing byproducts that are gas fermentation inhibitors. Such sulfur containing byproducts may be difficult to remove from the gas stream and may be passed to the bioreactor and harm the microorganisms. For example, while liquid dimethyl disulfide (DMDS) may be easy to handle and inject, use of DMDS to activate the metal based catalyst may result in sulfur containing byproducts such as H₂S, CS₂, COS. While H₂S is readily removed from the gas stream, CS₂ and COS are difficult and may be passed to the bioreactor in the treated gas stream. These sulfur containing compounds, especially CS₂, are harmful to the gas fermentation microorganisms. Instead of DMDS, H₂S may be used to activate the metal catalyst. Using H₂S to activate the metal catalyst does not result in the generation of sulfur containing byproducts such as CS₂ and COS, and thus eliminates potential fermentation inhibitors. Instead of DMDS liquid container and metering pump, a pressurized H₂S source or cylinder and a mass flow controller maybe used to control the injection of H₂S into the system for activation of the deoxygenation catalyst. Another source of suitable H₂S is tail gas from a fermentation reactor. For example, tail gas from a gas fermentation bioreactor may contain H₂S which then may be used for catalyst activation.

### Gas Sampling and Analytical System

To manage, maintain, and optimize the process, a robust analytical monitoring and control technology may be necessary. Such instruments may include, but are not limited to, a gas sampling system, and data logging/reporting software tools.

The analysis of the gas stream composition is a critical element of gas treatment. The analysis of the gas stream provides for the measurement and determination of which impurities need to be either converted and/or removed from the gas stream. To ensure that the gas stream has a desired composition, measurement of impurities in the gas stream at numerous points may be necessary. These measurements may be achieved through any suitable means, which may include online automatic monitoring, and may be completed in either a continuous and/or a periodic manner. In embodiments, the gas stream may be measured before and/or after being passed to the different removal beds.

In embodiments, the gas stream is measured prior to being passed to one or more removal beds. In certain instances, the measurement of the impurities present in the gas stream prior to being passed to the one or more removal beds determines which removal beds will be utilized. In embodiments, the determination of whether or not to utilize a hydrolysis bed is dependent on, at least in part, the measurement of the carbonyl sulfide (COS) present in the gas stream. In embodiments, the determination of whether or not to utilize a catalytic hydrogenation bed is dependent on, at least in part, the measurement of the acetylene (C₂H₂) present in the gas stream. In embodiments, the determination of whether or not to utilize a hydrogen cyanide removal bed is dependent on, at least in part, the measurement of the hydrogen cyanide (HCN) present in the gas stream.

The impurities present in the gas stream may vary based upon numerous factors. In certain embodiments, the impurities present in the gas stream are variable based upon the source from which the gas stream is derived. For example, gas streams sourced from a gasification process may have differing levels of impurities based upon changes in the substance being fed to the gasifier. In certain embodiments, the impurities present in the gas stream are variable based upon the gasifier operations. For example, gas streams sourced from gasification processes may have differing levels of impurities when plugging occurs in the gasifier.

In particular instances, the gas stream is obtained from a mixture of two or more sources. In various embodiments, the composition of the gas stream may be measured prior to, during, and/or after the sources are mixed.

In particular instances, the gas stream may be treated prior to, during, and/or after the sources are mixed. In certain instances, the composition of the gas stream is measured so as to analyze and determine which removal beds are necessary. This determination may be based on, at least in part, the one or more impurities present in the gas stream. In at least one instance, the composition of these gases may fluctuate over time resulting in varying proportions of impurities. These fluctuations may affect the performance of the treatment process. As such, it may be necessary to adjust the treatment process in response to the change in the composition. In various instances, this adjustment of the treatment process includes, the removal, bypassing, and/or addition of one or more removal bed. The selection of which removal bed to remove, bypass, and/or add may be due at least in part on the particular impurity present. In certain instances, one or more impurity previously not present, or present but below detection levels, may later be measured, which may then necessitate the addition of one or more removal bed. In certain instances, increased proportions of carbonyl sulfide (COS) and/or hydrogen cyanide (HCN) may necessitate the addition of a hydrolysis bed, whereas decreased proportions of carbonyl sulfide (COS) and/or hydrogen cyanide (HCN) may allow for the removal of the hydrolysis bed. In certain instances, increased proportions of carbon dioxide (CO₂), hydrogen sulfide (H₂S), and/or hydrogen cyanide (HCN) may necessitate the addition of an acid gas removal bed, whereas decreased proportions of carbon dioxide (CO₂), hydrogen sulfide (H₂S), and/or hydrogen cyanide (HCN) may allow for the removal of the acid gas removal bed. In certain instances, increased proportions of acetylene (C₂H₂) may necessitate the addition of a catalytic hydrogenation bed, whereas decreased proportions of acetylene (C₂H₂) may allow for the removal of the catalytic hydrogenation bed. In certain instances, increased proportions of oxygen (O₂) and/or acetylene (C₂H₂) may necessitate the addition of a deoxygenation bed, whereas decreased proportions of oxygen (O₂) and/or acetylene (C₂H₂) may allow for the removal of the deoxygenation bed.

For online measurement, each measurement point may be connected to the steel tubing to facilitate the transmission of the gas stream through the monitoring device. In embodiments, the gas stream is regulated by a pump device to provide a pressurized gas stream to the measurement device. In embodiments, the gas stream is pressurized between twenty and thirty pounds per square inch (138-207 kPa). In embodiments, different measurement devices are used to measure different impurities.

In embodiments, the level of the C₂H₂ and HCN levels in the gas stream is monitored by a spectrometer. In certain instances, the spectrometer will monitor the level of one or more of NH₃, CO₂, and/or H₂S in addition to C₂H₂ and/or HCN. In embodiments, the spectrometer is configured to measure at various sample points in periodic increments.

In embodiments, the hydrocarbons, BTEX, naphthalene, and the oxygenates dimethyl ether, diethyl ether, acetaldehyde, tetrahydrofuran, methyl ethyl ketone, acetone, methanol, and ethanol are measured by a gas chromatograph. In embodiments, the chromatograph is configured to measure at various sample points in periodic increments.

In embodiments, the nitrogen and sulfur in the gas stream are measured by a device which includes oxidative pyrolysis with Ultraviolet Fluorescence (UVF), and Chemiluminescence technologies. In embodiments, the device is configured to measure at various sample points in periodic increments.

In embodiments, bulk and/or trace impurities in the gas stream are measured by a gas chromatograph. Bulk and/or trace impurities may include but are not limited to, hydrogen, nitrogen, oxygen, methane, carbon monoxide, carbon dioxide, and hydrogen sulfide. In embodiments, the device is configured to measure at various sample points in periodic increments.

In embodiments, the various measurement devices may be connected to a software application, whereby the data collected by the measurement devices is interpreted and stored in a database. In embodiments, the data is parsed into an easily interpretable format, for example, a spreadsheet.

### Specialized Catalyst

A specialized catalyst, comprising copper supported on alumina, may be used to treat, and produce a fermentable gas stream from various gas sources. Such gas may be derived, in whole or in part from the combination of gas from one or more industrial process, synthesis gas, and/or producer gas. Specifically, it was found that this specialized catalyst was able to reduce oxygen, acetylene, and hydrogen cyanide such that oxygen is less than about 100 ppm, acetylene is less than about 1 ppm, and hydrogen cyanide is less than about 1 ppm in the fermentable gas stream. In various instances, the copper used for this catalyst was copper (I). In various instances, the copper used for this catalyst was reduced copper. The catalyst may be reduced by contacting with a hydrogen and nitrogen gas mixture at a desired temperature. The specialized catalyst may be employed as the sole catalyst in the gas treatment, or the specialized catalyst may be used as the deoxygenation catalyst.

To treat an input gas with high sulfur content, a sulfided version of the specialized catalyst may be employed. Sulfidation was achieved by passing a gas comprising a sulfidation reagent over a reduced version of the specialized catalyst. Such reduction and sulfidation can be carried out according to the prior art. In one embodiment, the sulfidation produced a sulfided copper (I) supported on alumina catalyst. In one embodiment, the sulfidation produced a sulfided copper (II) supported on alumina catalyst. The sulfided copper catalyst maybe especially useful at reducing the level of mercury (Hg) when present in the gas stream as coper sulfide is known to be an effective mercury adsorbent.

This catalyst is referred to below as an exemplary deoxygenation catalyst, b however it is understood that this particular catalyst may operate to remove more contaminates that oxygen.

### General

The particulars of this disclosure are especially suited to removal of contaminates since the amount of material being converted by the catalyst is very low as comparted to bulk conversion operations. Therefore, capacity levels of adsorbents are manageable, and exothermic temperature changes are manageable. Similarly, the target output levels are very low.

In embodiments, the treated fermentable gas stream is fed to a bioreactor containing at least one C1-fixing microorganism. The C1-fixing microorganism is capable of converting the treated fermentable C1-containing gas stream into useful chemicals and products through gas fermentation. To provide a noninhibiting fermentable gas stream to the bioreactor, the gas stream needs to contain a predetermined level of impurities or less. In embodiments, the impurities of concern include oxygen (O₂), hydrogen cyanide (HCN), acetylene (C₂H₂), BTEX (benzene, toluene, ethyl benzene, xylene), and sulfur (H₂S and COS). In various embodiments, the oxygen (O₂) level needs to be below one-hundred parts per million (100 ppm) to be at the predetermined level. In various embodiments, the hydrogen cyanide (HCN) needs to be below one part per million (1 ppm) to be at the predetermined level. The hydrogen cyanide (HCN) may be below one hundred parts per billion (100 ppb) to be at the predetermined level. In various embodiments, the acetylene (C₂H₂) needs to be below one part per million (1 ppm) to be at the predetermined level. In various embodiments, the BTEX needs to be below thirty parts per million (30 ppm) to be at the predetermined level. In various embodiments, the sulfur (H₂S and COS) needs to be below one part per million (1 ppm) to be at the predetermined level. In embodiments, all impurities must be at their predetermined levels to constitute a predetermined level of impurities.

The system may include further beds both prior to the hydrolysis bed and after the deoxygenation bed. These further beds may include but are not limited to, a particulate removal bed, a chloride removal bed, a tar removal bed, a hydrogen cyanide removal bed, and an additional acid gas removal bed, which may remove organics. In certain instances, a bed consisting of activated carbon is utilized to remove undesirable organic compounds. These organic compounds may, in certain instances, be formed by one or more removal bed. In embodiments, the gas is fed into the system to the beds in the following sequence: (1) particulate removal bed, (2) chloride removal bed, (3) tar removal bed, (4) hydrolysis bed, (5) acid gas removal bed, (5) catalytic hydrogenation bed, (6) deoxygenation bed, (7) hydrogen cyanide removal bed, and (8) additional acid gas removal bed. The beds may be arranged in modules or vessels and one or more bed may be co-located within a single module or vessel. It is also envisioned that a duplicate series of beds/vessel(s) may be employed in a lead-lag manner where one series of beds/vessel(s) is employed while the other is restored to operational condition. Periodically the two are switched so that the restored system is employed while the spent system is restored. It is also envisioned that multiple series of beds/vessel(s) may be employed.

The particulate removal bed may comprise any suitable bed capable of removing particulates from the gas stream. Particulates are typically associated with line plugging. In order to avoid line plugging, a particulate removal bed may be utilized. In embodiments, the particulate removal bed is a baghouse. The baghouse may be of any suitable type including, but not limited to, mechanical shakers, reverse gas, and pulse jet. In certain embodiments, the particulate removal bed is used prior to the other beds.

The chloride removal bed may comprise any suitable bed capable of removing chloride from the gas stream. Chloride is typically associated with corrosion in gas clean-up processes. To avoid corrosion, a chloride removal bed may be utilized. In embodiments, the chloride removal bed is a caustic scrubber capable of removing hydrogen chloride (HCl). In embodiments, the chloride removal bed is a cyclone capable of removing ammonium chloride (NH₄Cl).

The tar removal bed may comprise any suitable bed capable of removing tar from the gas stream. Tar may include but is not limited to, a heavy hydrocarbon such as naphthalene, which is typically associated with line plugging. In order to avoid line plugging, a tar removal bed may be utilized. In embodiments, the tar removal bed is an adsorption device. In certain instances, the adsorption device comprises activated carbon.

The hydrogen cyanide removal bed may comprise any suitable bed capable of removing hydrogen cyanide from the gas stream. Hydrogen cyanide is typically associated with inhibiting microorganisms. To avoid microorganism inhibition, a hydrogen cyanide removal bed may be utilized. In embodiments, the hydrogen cyanide removal bed is a copper treated activated carbon device.

The additional acid gas removal bed may comprise any suitable bed capable of removing carbon dioxide from the gas stream. High levels of carbon dioxide may dilute the gas stream, thus requiring larger bioreactors and/or additional fermentation trains. In order to avoid gas stream dilution by the carbon dioxide, an additional acid gas removal bed may be utilized. In embodiments, the additional acid gas removal bed is a PSA bed, which may utilize calcium hydroxide.

The system may include one or more temperature modules to either increase or decrease the temperature of the gas stream. These temperature modules may be placed before and/or after other modules to increase or decrease the temperature of the gas stream between modules. The temperature modules may comprise any suitable module capable of increasing or decreasing the temperature of the gas stream. In embodiments, the temperature modules are a shell and tube heat exchanger. The shell tube heat exchanger comprises a shell with a bundle of tubes inside the shell. The shell and tube heat exchanger is capable of regulating the temperature of the gas stream by passing a fluid, for example water, through the shell, while simultaneously passing the gas stream through the bundle of tubes. The heat is transferred between the gas stream and the fluid through the tube walls.

The system may include pressure modules to either increase or decrease the pressure of the gas stream. These pressure modules may be placed before and/or after other modules. The pressure modules may comprise any suitable module capable of increasing or decreasing the pressure of the gas stream. In embodiments, the pressure module is a compressor. The compressor is capable of increasing the pressure of the gas stream to a value that is suitable for the transferring of the gas stream. In embodiments, the pressure module is a valve. The valve is capable of decreasing the pressure of the gas stream to a value that is suitable for the transferring of the gas stream.

In a revamp situation, where limited vessels or temperature modules are available, at least two or more beds may be co-located within a single vessel, and a single temperature module such as a heat exchanger may be used to bring the single vessel to a common operating temperature for all the beds contained therein. In one example, the hydrocarbon removal bed, the hydrolysis bed, the sulfur guard bed, and the deoxygenation bed are all co-located within a single vessel. In another example, the hydrocarbon removal bed is in an independent module while the hydrolysis bed, the sulfur guard bed, and the deoxygenation bed are co-located in a single vessel. In both examples, the specialized copper-based catalyst discussed above may be used in the deoxygenation bed, or another deoxygenation catalyst may be used.

When co-locating multiple beds in a single vessel, all beds must operate at a common temperature. This may be challenging since typically adsorbents and catalysts operate at different temperatures. When using the specialized copper-based catalyst discussed above, the catalyst may need to be reduced before use. Therefore, the catalyst may need to be brought to from 150° C. to 200° C., or 225° C. or 250° C. to reduce the copper component. Because all beds are in the same vessel, the other beds should withstand the same temperature as the reduction temperature of the deoxygenation catalyst without changing form. An advantageous choice for the sulfur guard bed is a zinc oxide adsorbent since the zinc oxide only undergoes reduction at a temperature far greater than the reduction temperature of the deoxygenation catalyst. If the zine oxide were to be reduced, it would no longer absorb sulfur and not function as a sulfur guard bed. In other words, the process is operated at a temperature below the reduction temperature of the sulfur guard bed adsorbent.

As typical deoxygenation catalysts are not always sulfur tolerant, the sulfur guard bed may be located upstream of the deoxygenation catalyst to protect the deoxygenation catalyst from exposure to sulfur compounds.

The individual beds that are co-located, or stacked, within a single vessel may be separated from one another by screens. It is advantageous to use different particle sizes for the different beds so that sieving may be used after the vessel is unloaded for replacement of spent adsorbent. Catalyst may be separated from spend adsorbent and potentially regenerated and reused. It is also contemplated that various catalysts may be mixed in a single bed, so long as the sulfur guard bed is located upstream of the deoxygenation catalyst.

Removing stacked beds from the vessel may involve using gravity to allow one or more beds to flow out of the vessel or using vacuum to pull one or more beds out of the vessel. The beds may be made up of individual containers which may be removed from the vessel separately and regenerated, reloaded with fresh material, or simply placed back into the vessel after other beds have been regenerated or reloaded. The unloading of spent adsorbent potentially along with other beds within the vessel is designed to be accomplished in one working shift of an industrial plant. Different particle sizes for each bed may be employed so that upon unloading from the vessel, the spend adsorbent is readily separated by sieves from the catalysts. The catalysts may then be reloaded and reused. Fresh adsorbent may be reloaded, replacing the spend adsorbent. It is not required that that recovered catalysts are highly pure, in other words it is acceptable if the sieving technique results in a percentage of the catalyst to be mixed from one bed to another bed.

To aid in fluid distribution, ceramic balls may be placed at the top of the vessel.

In an alternative arrangement to a stacked bed system, the different beds may be arranged in nesting baskets so that each may be removed from the vessel and the basket holding the spent adsorbent may be emptied and reloaded with fresh adsorbent without losing catalyst in the other baskets. The vessel in this embodiment would be operated with concentric radial flow of the gas. The inner most basket may contain the hydrolysis catalyst, the middle concentric basket would be zinc oxide, and the outer concentric basket would be the specialized copper based deoxygenation catalyst.

In an alternative embodiment, two functionalities may be combined into a single material, or two materials may be physically mixed to form a single bed. For example, the hydrolysis function and the sulfur adsorption function may be combined into a single material bifunctional such zinc oxide supported on an alumina support. The single vessel may then contain two beds, a combined hydrolysis and sulfur guard bed such as zinc oxide supported on an alumina support followed by a deoxygenation bed using a deoxygenation catalyst such as the specialized copper based catalyst.

In one embodiment the beds of adsorbent or catalyst are ordered in an advantageous order. The hydrolysis bed is positioned upstream of the sulfur guard bed so that sulfur containing compounds are converted by the hydrolysis catalyst, such as alumina, to a reduced form of sulfur prior to contact with the sulfur guard bed and adsorption by the sulfur guard bed material such as zinc oxide. The sulfur guard bed is positioned upstream of the deoxygenation catalyst bed so that sulfur containing compounds are removed prior to the gas contacting the deoxygenation catalyst which are often susceptible to sulfur poisoning.

In another embodiment, the vessel may be externally headed at locations near to the deoxygenation bed without heating the input gas. The reactions occurring at the hydrolysis bed may be exothermic and therefor auto-heating that portion of the vessel. Heat would only need to be applied to the portion of the vessel downstream of the hydrolysis bed.

In yet another embodiment, a screen or other physical barrier may be employed above the sulfur guard bed in the vessel, so that upon gravity driven unloading of spent adsorbent from the vessel, beds located above the physical barrier are retained within the vessel. Multiple physical barriers may be employed to control gravity removal of different beds. A retained bed may be vacuum removed, or gravity removed.

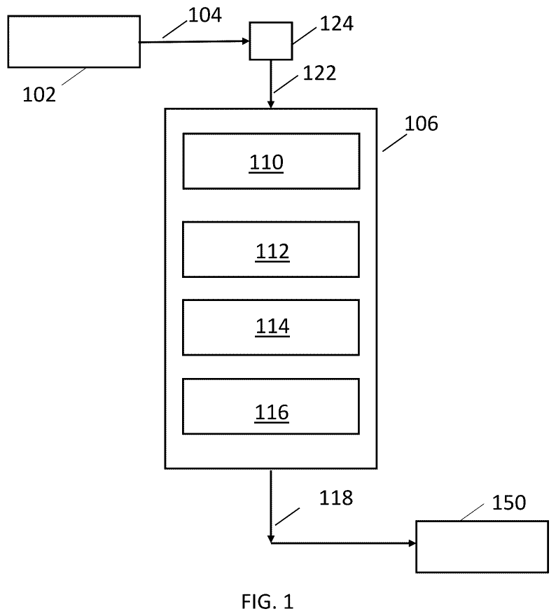

FIG. 1 shows a system for selectively removing impurities from an input gas stream 104, the system comprises a hydrocarbon removal bed 110, a hydrolysis bed 112, a sulfur guard bed 114, a deoxygenation bed 116, and a bioreactor 150. The hydrocarbon removal bed 110, hydrolysis bed 112, sulfur guard bed 114, and deoxygenation bed 116 are all contained within vessel 106. The gas stream may be derived from any industrial, producer, and/or synthesis gas source 102. Prior to entering vessel 106, input gas may be heated to the operating temperature of vessel 106, which may be from about 175° C. to about 250° C. or from about 200° C. to 300° C. or from about 300° C. to 500° C. or 550° C. Input gas stream 104 is fed from the industrial, producer, and/or synthesis gas source 102 to heater 124, which can be a heat exchanger. Heated input gas 122 is passed to vessel 106. Within vessel 106, input gas sequentially contacts hydrocarbon removal bed 110, hydrolysis bed 112, sulfur guard bed 114, deoxygenation bed 116.

Contact with hydrogen removal bed containing an adsorbent such as activated charcoal results in one or more hydrocarbons being adsorbent onto the activated charcoal and removed from the gas stream. Contact with hydrolysis bed 112 converts at least one impurity in the gas stream, to provide a post-hydrolysis gas stream. Hydrogen cyanide (HCN) and carbonyl sulfide (COS) are two anticipated constituents that maybe chemically reacted with water and to be removed from the gas stream. If COS is present is advantageous to convert COS to hydrogen sulfide (H₂S) which then may be removed in the following sulfur guard bed 114.

Now that HCN has been removed and COS converted to H₂S, the gas passes to sulfur guard bed 114 where H₂S is adsorbed and removed from the gas. Then the gas passes to deoxygenation bed 116 where oxygen (O₂) is reduced to either carbon dioxide (CO₂) or water (H₂O) and thereby removed from the gas. The treated gas exits vessel 106 into treated gas steam 118 which is passed to bioreactor 150 for fermentation. The bioreactor may contain C1-fixing microorganisms capable of producing products and a post-fermentation gaseous substrate from the gas stream.

In one embodiment, the hydrocarbon removal bed 110 comprises activated charcoal, hydrolysis bed 112 comprises a hydrolysis catalyst, sulfur guard bed 114 comprises zinc oxide, and deoxygenation bed 116 comprises the un-sulfided specialized copper based deoxygenation catalyst discussed above. This selection of materials is surprisingly effective since the other materials in beds other than deoxygenation bed 116 are not affected by the reduction gas and reduction temperature needed to reduce the specialized copper based deoxygenation catalyst. Furthermore, at the operating conditions of the process, the zinc oxide will not reduce to zinc. If the zinc oxide were to be reduced to zinc, it would become ineffective in adsorbing sulfur containing compounds and then the deoxygenation catalyst may be poisoned by sulfur containing catalysts. If the deoxygenation catalyst fails to adequately remove sufficient oxygen, anaerobic microorganism(s) in the fermentation bioreactor may be harmed.

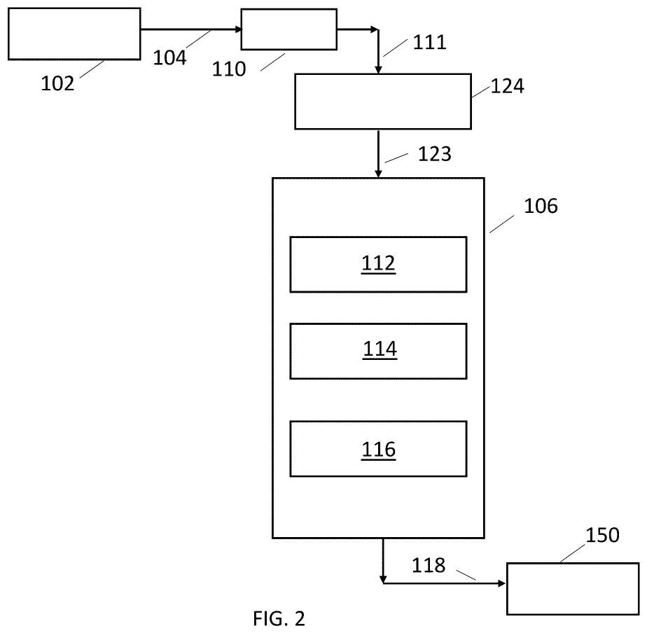

Turing to FIG. 2, the system is similar to FIG. 1 except that the hydrocarbon removal bed is not located within the vessel but is instead located in a module upstream of the vessel. The hydrocarbon removal bed module 110 may be upstream of heating device 124 as shown in FIG. 2 or may be downstream of heating device 124 (not shown). FIG. 2 is a system for selectively removing impurities from an input gas stream 104, the system comprises a hydrocarbon removal bed 110, a hydrolysis bed 112, a sulfur guard bed 114, a deoxygenation bed 116, and a bioreactor 150. The hydrolysis bed 112, sulfur guard bed 114, and deoxygenation bed 116 are all contained within vessel 106. Hydrocarbon removal bed 110 is located upstream of vessel 106. The input gas stream may be derived from any industrial, producer, and/or synthesis gas source 102. Input gas stream 104 is contacted with a hydrocarbon removal adsorbent in module 110 to remove hydrocarbons from the input gas stream and generate hydrocarbon depleted input gas stream 111. In one embodiment the hydrocarbon removal adsorbent is activated charcoal. Prior to entering vessel 106, hydrocarbon depleted input gas 111 may be heated in heating device 124 to the operating temperature of vessel 106, which may be from about 175° C. to about 250° C. or from about 200° C. to 300° C. or from about 300° C. to 500° C. or 550° C. Heating device 124 may be a heater or a heat exchanger. It is advantageous to repurpose a heating device that is readily available. Heated input gas 123 is passed to vessel 106. Within vessel 106, input gas sequentially contacts hydrolysis bed 112, sulfur guard bed 114, deoxygenation bed 116.

Contact with hydrogen removal bed containing an adsorbent such as activated charcoal results in one or more hydrocarbons being adsorbent onto the activated charcoal and removed from the gas stream. Contact with hydrolysis bed 112 converts at least one impurity in the gas stream, to provide a post-hydrolysis gas stream. Hydrogen cyanide (HCN) and carbonyl sulfide (COS) are two anticipated constituents that maybe chemically reacted with water and to be removed from the gas stream. If COS is present is advantageous to convert COS to hydrogen sulfide (H₂S) which then may be removed in the following sulfur guard bed 114.

Now that HCN has been removed and COS converted to H₂S, the gas passes to sulfur guard bed 114 where H₂S is adsorbed and removed from the gas. Then the gas passes to deoxygenation bed 116 where oxygen (O₂) is reduced to either carbon dioxide (CO₂) or water (H₂O) and thereby removed from the gas. The treated gas exits vessel 106 into treated gas steam 118 which is passed to bioreactor 150 for fermentation. The bioreactor may contain C1-fixing microorganisms capable of producing products and a post-fermentation gaseous substrate from the gas stream.

In one embodiment, the hydrocarbon removal bed 110 comprises activated charcoal, hydrolysis bed 112 comprises a hydrolysis catalyst such as alumina, sulfur guard bed 114 comprises zinc oxide, and deoxygenation bed 116 comprises the non-sulfided specialized copper based deoxygenation catalyst discussed above. This selection of materials is surprisingly effective since materials in beds other than deoxygenation bed 116 are not affected by the reduction gas and reduction temperature needed to reduce the specialized copper based deoxygenation catalyst.

As discussed earlier, in one embodiment, hydrolysis bed 112 and sulfur guard bed 114 may be a physical mixture and combined into a single bed. In another embodiment, the functionality of the hydrolysis catalyst and sulfur adsorbent may be combined into a composite material such as zinc oxide on an alumina support, and the composite material used as a single bed performing the function of both debs 112 and 114.

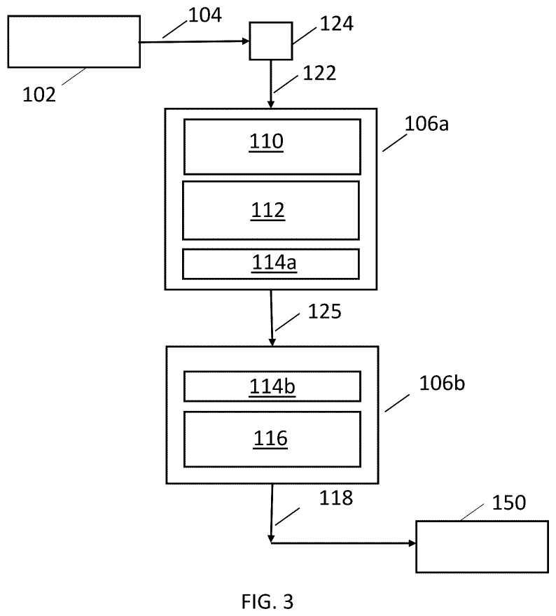

FIG. 3 is similar to FIG. 1, except that vessel 106 is replaced by two vessels, first vessel 106a and second vessel 106b. First vessel 106a contains the hydrocarbon removal bed 110, hydrolysis bed 112 comprising a hydrolysis catalyst, and a first sulfur guard bed 114a. Second vessel 106b contains a second sulfur guard bed 114b and a deoxygenation bed 116. The adsorbent in sulfur guard bed 114a and 114b may be the same or different. Effluent from first vessel 116a passes to second vessel 106b through line 125.

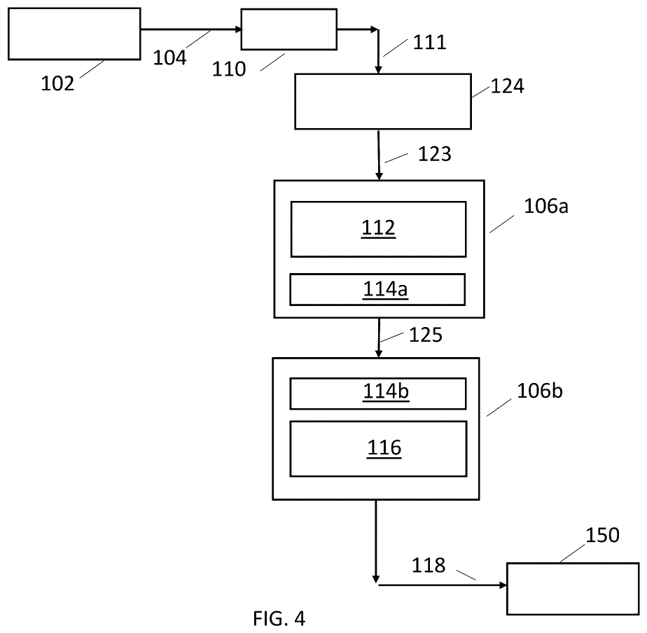

FIG. 4 is similar to FIG. 2, except that vessel 106 is replaced by two vessels, first vessel 106a and second vessel 106b. First vessel 106a contains the hydrolysis bed 112 comprising a hydrolysis catalyst, and a first sulfur guard bed 114a. Second vessel 106b contains a second sulfur guard bed 114b and a deoxygenation bed 116. The adsorbent in sulfur guard bed 114a and 114b may be the same or different. Effluent from first vessel 116a passes to second vessel 106b through line 125.

### Example 1

A gas cleaning system was configured to receive a blended gas stream. The blended gas stream being designed to represent a stream received from a steel mill. The gas cleaning system incorporated the following beds in the following order: (i) hydrolysis bed, (ii) acid gas removal bed, (iii) catalytic hydrogenation bed, and (iv) deoxygenation bed. The hydrolysis bed consisting of a bed of gamma-alumina adsorbent (BASF F-200). The acid gas removal bed consisting of a bed of zinc oxide adsorbent (RCI ZOP-116). The catalytic hydrogenation bed consisting of palladium on alumina catalyst (BASF R0-20/47). The deoxygenation bed consisting of a copper catalyst (BASF CU0226S).

Prior to testing the substrate, the hydrogenation catalyst was reduced in 1% H₂ by volume in N₂ at 120° C. for at least 12 hours. The deoxygenation catalyst was reduced in 1% H₂ by volume in N₂ at 250° C. for at least 12 hours.

The composition of the blended gas stream being fed to the gas cleaning system is illustrated by the below table.

| Compound | | |
|---|---|---|
| Hydrogen | 6.8% | by volume |
| Carbon Monoxide | 30.6% | by volume |
| Carbon Dioxide | 18.4% | by volume |
| Nitrogen | 43.0% | by volume |
| Water | 4500 | ppm |
| Oxygen | 6700 | ppm |
| Acetylene | 500 | ppm |
| Hydrogen Cyanide | 60 | ppm |

In addition to the above compounds, trace levels of methane and dimethyl ether were detected in the blended stream. These compounds are impurities in the feed gas.

These rate at which the gas stream was fed and the inlet temperature of each bed is illustrated by the below table. The pressure of each bed was 345 kPag.

| | Gas Hourly | Bed Inlet |
|---|---|---|
| | Space Velocity | Temperature |
| Bed | (GHSV) Hour⁻¹ | (° C.) |
| Hydrolysis | 2000 | 200 |
| Acid Gas Removal | 370 | 20 |
| Catalytic | 5500 | 120 |
| Hydrogenation | | |
| Deoxygenation | 4000 | 200 |

This configuration successfully produced a fermentable gas stream. Target contaminant removal was achieved. The composition of the fermentable gas stream is illustrated by the below table.

| Compound | | |
|---|---|---|
| Oxygen | 0.50 | ppm |
| Acetylene | 0.062 | ppm |
| Hydrogen Cyanide | <0.010 | ppm |

Trace amounts of methane were detected in the fermentable gas stream. However, the amount of methane in the outlet stream was similar to the amount of methane detected in the inlet stream, thus no production of methane was detected. Trace ethane and ethylene were detected. Ethane and ethylene are products from acetylene removal and are not microorganism inhibitors. No other impurities were detected in the outlet stream using this configuration. No microorganism inhibitors were formed using this configuration.

The outlet concentration of the CO was 30.1% by volume. This outlet concentration corresponds to 2.6% consumption of the input CO, which is well below a maximum consumption of 10%.

### Example 2

A gas cleaning system, similar to Example 1, was configured to receive a blended gas stream. The blended gas stream being designed to represent a stream received from a steel mill. The gas cleaning system incorporated the following beds in the following order: (i) hydrolysis bed, (ii) acid gas removal bed, and (iii) deoxygenation bed. The hydrolysis bed consisting of a bed of gamma-alumina adsorbent (BASF F-200). The acid gas removal bed consisting of a bed of zinc oxide adsorbent (RCI ZOP-116). The deoxygenation bed consisting of a copper catalyst (BASF CU0226S).

The composition of the blended gas stream being fed to the gas cleaning system is illustrated by the below table.

| Compound | | |
|---|---|---|
| Hydrogen | 6.8% | by volume |
| Carbon Monoxide | 30.6% | by volume |
| Carbon Dioxide | 18.4% | by volume |
| Nitrogen | 43.0% | by volume |
| Water | 4500 | ppm |
| Oxygen | 6700 | ppm |
| Acetylene | 500 | ppm |
| Hydrogen Cyanide | 60 | ppm |

In addition to the above compounds, trace levels of methane were detected in the blended stream. These compounds are impurities in the feed gas.

These rate at which the gas stream was fed and the inlet temperature of each bed is illustrated by the below table. The pressure of each bed was 345 kPag.

| | Gas Hourly | Bed Inlet |
|---|---|---|
| | Space Velocity | Temperature |
| Bed | (GHSV) Hour⁻¹ | (° C.) |
| Hydrolysis | 2000 | 200 |
| Acid Gas Removal | 370 | 20 |
| Deoxygenation | 4000 | 200 |

This configuration successfully produced a fermentable gas stream. Target contaminant removal was achieved. The composition of the fermentable gas stream is illustrated by the below table.

| Compound | | |
|---|---|---|
| Oxygen | 0.45 | ppm |
| Acetylene | 0.065 | ppm |
| Hydrogen Cyanide | <0.010 | ppm |

Trace amounts of methane were detected in the fermentable gas stream. However, the amount of methane in the outlet stream was similar to the amount of methane detected in the inlet stream, thus no production of methane was detected. Trace ethane and ethylene were detected. Ethane and ethylene are products from acetylene removal. Trace amounts of dimethyl ether and acetaldehyde were detected. Dimethyl ether and acetaldehyde are not microorganism inhibitors. No microorganism inhibitors were formed using this configuration.

Trace amounts of dimethyl ether and acetaldehyde were removed by passing the fermentable gas stream to an organic compound removal bed. The flowrate of the gas stream to the organic compound removal bed was such that the gas hourly space velocity was 370 hr.⁻¹.

The outlet concentration of the CO was 29.8% by volume. This outlet concentration corresponds to 4.0% consumption of the input CO, which is well below a maximum consumption of 10%.

In addition to running the gas cleaning system at 345 KPag, using this configuration and this gas composition, the pressure was increased such that the pressure of each bed was 690 kPag in order to evaluate how pressure may affect the system.

It was found that at increased pressure (690 kPag for each bed), the configuration successfully produced a fermentable gas stream. Target contaminant removal was achieved. The composition of the fermentable gas stream is illustrated by the below table.

| Compound | | |
|---|---|---|
| Oxygen | 0.41 | ppm |
| Acetylene | 0.076 | ppm |
| Hydrogen Cyanide | <0.010 | ppm |

Trace amounts of methane were detected in the fermentable gas stream. However, the amount of methane in the outlet stream was similar to the amount of methane detected in the inlet stream, thus no production of methane was detected. Trace ethane and ethylene were detected. Ethane and ethylene are products from acetylene removal and are not microorganism inhibitors. Trace amounts of dimethyl ether and acetaldehyde were detected. Dimethyl ether and acetaldehyde are not microorganism inhibitors. No impurities were detected in the outlet stream using this configuration.

Trace amounts of dimethyl ether and acetaldehyde were removed by passing the fermentable gas stream to an organic compound removal bed. The flowrate of the gas stream to the organic compound removal bed was such that the gas hourly space velocity was 370 hr.⁻¹.

The outlet concentration of the CO was 29.8% by volume. This outlet concentration corresponds to 3.3% consumption of the input CO, which is well below a maximum consumption of 10%.

### Example 3

A gas cleaning system was configured to receive a blended gas stream. The blended gas stream being designed to represent a stream received from a steel mill. The gas cleaning system incorporated the following beds in the following order: (i) hydrolysis bed, (ii) acid gas removal bed, (iii) catalytic hydrogenation bed, (iv) deoxygenation bed, and (v) organic compound removal bed. The hydrolysis bed consisting of a bed of gamma-alumina adsorbent (BASF F-200). The acid gas removal bed consisting of a bed of zinc oxide adsorbent (RCI ZOP-116). The catalytic hydrogenation bed consisting of palladium on alumina catalyst (BASF R0-20/47). The deoxygenation bed consisting of a copper catalyst (BASF Cu0226S).

Prior to testing the substrate, the hydrogenation catalyst was reduced in 1% H₂ by volume in N₂ at 120° C. for at least 12 hours. The deoxygenation catalyst was reduced in 1% H₂ by volume in N₂ at 250° C. for at least 12 hours.

The composition of the blended gas stream being fed to the gas cleaning system is illustrated by the below table.

| Compound | | |
|---|---|---|
| Hydrogen | 6.2% | by volume |
| Carbon Monoxide | 27.6% | by volume |
| Carbon Dioxide | 16.2% | by volume |
| Nitrogen | 49.1% | by volume |
| Water | 2400 | ppm |
| Hydrogen Sulfide | 40.0 | ppm |
| Carbonyl Sulfide | 4.0 | ppm |
| Oxygen | 6000 | ppm |
| Acetylene | 550 | ppm |
| Hydrogen Cyanide | 20 | ppm |

In addition to the above compounds, trace levels of methane were detected in the blended stream. These compounds are impurities in the feed gas.

These rate at which the gas stream was fed and the inlet temperature of each bed is illustrated by the below table. The pressure of each bed was 690 kPag.

| | Gas Hourly Space | Bed Inlet |
|---|---|---|
| Bed | Velocity (GHSV) Hour⁻¹ | Temperature (° C.) |
| Hydrolysis | 2000 | 200 |
| Acid Gas Removal | 370 | 20 |
| Catalytic Hydrogenation | 5500 | 120 |
| Deoxygenation | 4000 | 200 |
| Organic Removal | 370 | 20 |

This configuration successfully produced a fermentable gas stream. Target contaminant removal was achieved. The composition of the fermentable gas stream is illustrated by the below table.

| Compound | | |
|---|---|---|
| Oxygen | 0.38 | ppm |
| Acetylene | 0.168 | ppm |
| Hydrogen Cyanide | <0.030 | ppm |

Trace amounts of methane were detected in the fermentable gas stream. However, the amount of methane in the outlet stream was similar to the amount of methane detected as an impurity in the inlet stream, thus no production of methane was detected. Trace ethane and ethylene were detected. Ethane and ethylene are products from acetylene removal and are not microbial inhibitors. No other impurities were detected in the outlet stream using this configuration. No microbial inhibitors were formed using this configuration of beds.

The outlet concentration of the CO was 26.6% by volume. This outlet concentration corresponds to 3.8% consumption of the input CO, which is well below a maximum consumption of 10%.

### Example 4

A gas cleaning system was configured similarly to Example 3 to receive a blended gas stream. The blended gas stream being designed to represent a stream received from a steel mill. The gas cleaning system incorporated the following beds in the following order: (i) hydrolysis bed, (ii) acid gas removal bed, (iii) deoxygenation bed, and (iv) organic compound removal bed. The hydrolysis bed consisting of a bed of gamma-alumina adsorbent (BASF F-200). The acid gas removal bed consisting of a bed of zinc oxide adsorbent (RCI ZOP-116). The deoxygenation bed consisting of a copper catalyst (BASF Cu0226S).

The composition of the blended gas stream being fed to the gas cleaning system is illustrated by the below table.

| Compound | | |
|---|---|---|
| Hydrogen | 6.2% | by volume |
| Carbon Monoxide | 27.6% | by volume |
| Carbon Dioxide | 16.2% | by volume |
| Nitrogen | 49.1% | by volume |
| Water | 2400 | ppm |
| Hydrogen Sulfide | 40.0 | ppm |
| Carbonyl Sulfide | 4.0 | ppm |
| Oxygen | 6000 | ppm |
| Acetylene | 550 | ppm |
| Hydrogen Cyanide | 20 | ppm |

In addition to the above compounds, trace levels of methane were detected in the blended stream. These compounds are impurities in the feed gas.

These rate at which the gas stream was fed and the inlet temperature of each bed is illustrated b the below table. The pressure of each bed was 690 kPag.

| | Gas Hourly Space | Bed Inlet |
|---|---|---|
| Bed | Velocity (GHSV) Hour⁻¹ | Temperature (° C.) |
| Hydrolysis | 2000 | 200 |
| Acid Gas Removal | 370 | 20 |
| Deoxygenation | 4000 | 200 |
| Organic Removal | 370 | 20 |

This configuration successfully produced a fermentable gas stream. Target contaminant removal was achieved. The composition of the fermentable gas stream is illustrated by the below table.

| Compound | | |
|---|---|---|
| Oxygen | 0.34 | ppm |
| Acetylene | 0.073 | ppm |
| Hydrogen Cyanide | <0.010 | ppm |

Trace amounts of methane were detected in the fermentable gas stream. However, the amount of methane in the outlet stream was similar to the amount of methane detected as an impurity in the inlet stream, thus no production of methane was detected. Trace ethane and ethylene were detected. Ethane and ethylene are products from acetylene removal and are not microbial inhibitors. No other impurities were detected in the outlet stream using this configuration. No microbial inhibitors were formed using this configuration of beds. The outlet concentration of the CO was 26.2% by volume. This outlet concentration corresponds to 4.9% consumption of the input CO, which is well below a maximum consumption of 10%.

### Example 5

A gas cleaning system, similar to Example 2, was configured to receive a blended gas stream. The blended gas stream has higher concentrations of microbial inhibitors. The concentrations being in the range that is expected from biomass or municipal solid waste gasification or treated coke oven gas. The gas cleaning system incorporated the following beds in the following order: (i) hydrolysis bed, (ii) acid gas removal bed, (iii) deoxygenation bed, and (iv) organic compound removal bed. The hydrolysis bed consisting of a bed of gamma-alumina adsorbent (BASF F-200). The acid gas removal bed consisting of a bed of zinc oxide adsorbent (RCI ZOP-116). The deoxygenation bed consisting of a copper catalyst (BASF Cu 0226S).

Prior to testing the substrate, the deoxygenation catalyst was reduced in 1% H₂ by volume in N₂ at 250° C. for at least 12 hours.

The composition of the blended gas stream being fed to the gas cleaning system is illustrated by the below table.

| Compound | | |
|---|---|---|
| Hydrogen | 4.1% | by volume |
| Carbon Monoxide | 17.8% | by volume |
| Carbon Dioxide | 10.7% | by volume |
| Nitrogen | 66.3% | by volume |
| Water | 2000 | ppm |
| Oxygen | 7600 | ppm |
| Acetylene | 860 | ppm |
| Hydrogen Cyanide | 280 | ppm |

In addition to the above compounds, trace levels of methane were detected in the blended stream. This compound is an impurity in the feed gas.

These rate at which the gas stream was fed and the inlet temperature of each bed is illustrated by the below table. The pressure of each bed was 690 kPag.

| | Gas Hourly Space | Bed Inlet |
|---|---|---|
| Bed | Velocity (GHSV) Hour⁻¹ | Temperature (° C.) |
| Hydrolysis | 2000 | 200 |
| Acid Gas Removal | 370 | 20 |
| Deoxygenation | 4000 | 200 |
| Organic Removal | 370 | 20 |

This configuration successfully produced a fermentable gas stream. Target contaminant removal was achieved. The composition of the fermentable gas stream is illustrated by the below table.

| Compound | | |
|---|---|---|
| Oxygen | 0.46 | ppm |
| Acetylene | 0.040 | ppm |
| Hydrogen Cyanide | <0.010 | ppm |

Trace amounts of methane were detected in the fermentable gas stream. However, the amount of methane in the outlet stream was similar to the amount of methane detected as an impurity in the inlet stream, thus no production of methane was detected. Trace ethane and ethylene were detected. Ethane and ethylene are products from acetylene removal and are not microbial inhibitors. No other impurities were detected in the outlet stream using this configuration. No microbial inhibitors were formed using this configuration of beds.

The outlet concentration of the CO was 16.6% by volume. This outlet concentration corresponds to 6.8% consumption of the input CO, which is well below a maximum consumption of 10%.

### Example 6

A gas cleaning system was configured to receive a blended gas stream. The blended gas stream being designed to represent a stream received from a steel mill. The gas cleaning system incorporated only one bed. The bed consisted of a copper catalyst (BASF Cu0226S).

Prior to testing a substrate, the deoxygenation catalyst was reduced in 1% H₂ by volume in N₂ at 250° C. for at least 12 hrs.

The composition of the blended gas stream being fed to the gas cleaning system is illustrated by the below table.

| Compound | | |
|---|---|---|
| Hydrogen | 7.0% | by volume |
| Carbon Monoxide | 31.6% | by volume |
| Carbon Dioxide | 18.5% | by volume |
| Nitrogen | 41.9% | by volume |
| Water | 4500 | ppm |
| Oxygen | 5900 | ppm |
| Acetylene | 490 | ppm |
| Hydrogen Cyanide | 20 | ppm |

In addition to the above compounds, trace levels of methane were detected in the blended stream. This compound is an impurity in the feed gas.

The rate at which the gas stream was fed corresponds to a 4000 hr-1 gas hourly space velocity. The inlet temperature of the bed was 200° C. The pressure of the bed was 690 kPag.

This bed successfully produced a fermentable gas stream. Target contaminant removal was achieved. The composition of the fermentable gas stream is illustrated by the below table.

| Compound | | |
|---|---|---|
| Oxygen | 0.41 | ppm |
| Acetylene | 0.060 | ppm |
| Hydrogen Cyanide | <0.010 | ppm |

Trace amounts of methane were detected in the fermentable gas stream. However, the amount of methane in the outlet stream was similar to the amount of methane detected as an impurity in the inlet stream, thus no production of methane was detected. Trace ethane and ethylene were detected. Ethane and ethylene are products from acetylene removal and are not microbial inhibitors. Methanol was detected in the fermentable gas stream. Methanol is not a microbial inhibitor. No other impurities were detected in the outlet stream using this configuration. No microbial inhibitors were formed using this configuration of beds.

The outlet concentration of the CO was 30.2% by volume. This outlet concentration corresponds to 4.2% consumption of the input CO, which is well below a maximum consumption of 10%.

### Example 7

A gas cleaning system was configured to receive a blended gas stream. The blended gas stream being designed to represent a stream received from a steel mill. The gas cleaning system incorporated only one bed. The bed consisted of a copper catalyst (BASF Cu0226S).

Prior to testing a substrate, the catalyst was reduced in 1% H₂ by volume in N₂ at 250° C. for at least 12 hrs. Following the catalyst reduction, the catalyst was sulfided using a gas stream of 1% H₂S by volume, 5% H₂ by volume in N₂. The catalyst was sulfided at 220° C. for 18 hours.

The composition of the blended gas stream being fed to the gas cleaning system is illustrated by the below table.

| Compound | | |
|---|---|---|
| Hydrogen | 6.1% | by volume |
| Carbon Monoxide | 27.2% | by volume |
| Carbon Dioxide | 16.0% | by volume |
| Nitrogen | 49.8% | by volume |
| Water | 2400 | ppm |
| Hydrogen Sulfide | 39 | ppm |
| Carbonyl Sulfide | 4.0 | ppm |
| Oxygen | 6200 | ppm |
| Acetylene | 550 | ppm |
| Hydrogen Cyanide | 19 | ppm |

In addition to the above compounds, trace levels of methane were detected in the blended stream. This compound is an impurity in the feed gas.

The rate at which the gas stream was fed corresponds to a 2000 hr-1 gas hourly space velocity. The inlet temperature of the bed was 280° C. The pressure of the bed was 690 kPag.

This bed successfully produced a fermentable gas stream. Target contaminant removal was achieved. The composition of the fermentable gas stream is illustrated by the below table.

| Compound | | |
|---|---|---|
| Oxygen | 0.42 | ppm |
| Acetylene | 0.581 | ppm |
| Hydrogen Cyanide | 0.011 | ppm |

Trace amounts of methane were detected in the fermentable gas stream. However, the amount of methane in the outlet stream was similar to the amount of methane detected as an impurity in the inlet stream, thus no production of methane was detected. Trace ethane and ethylene were detected. Ethane and ethylene are products from acetylene removal and are not microbial inhibitors. Acetaldehyde was detected in the fermentable gas stream. Acetaldehyde is not a microbial inhibitor. No other impurities were detected in the outlet stream using this configuration. No microbial inhibitors were formed using this configuration of beds.

The outlet concentration of the CO was 26.9% by volume. This outlet concentration corresponds to 1.0% consumption of the input CO, which is well below a maximum consumption of 10%.

### Example 8

A gas cleaning system similar to Example 7 was configured to receive a blended gas stream. The blended gas stream comprised higher concentrations of microbial inhibitors. The concentrations being in the range expected from biomass or municipal solid waste gasification or treated coke oven gas. The gas cleaning system incorporated only one bed. The bed consisted of a copper catalyst (BASF Cu0226S).

The composition of the blended gas stream being fed to the gas cleaning system is illustrated by the below table.

| Compound | | |
|---|---|---|
| Hydrogen | 3.8% | by volume |
| Carbon Monoxide | 16.4% | by volume |
| Carbon Dioxide | 9.1% | by volume |
| Nitrogen | 69.6% | by volume |
| Water | 2200 | ppm |
| Hydrogen Sulfide | 40 | ppm |
| Carbonyl Sulfide | 4 | ppm |
| Oxygen | 6600 | ppm |
| Acetylene | 1060 | ppm |
| Hydrogen Cyanide | 400 | ppm |

In addition to the above compounds, trace levels of methane were detected in the blended stream. This compound is an impurity in the feed gas.

The rate at which the gas stream was fed corresponds to a 1000 hr-1 gas hourly space velocity. The inlet temperature of the bed was 300° C. The pressure of the bed was 690 kPag.

This bed successfully produced a fermentable gas stream. Target contaminant removal was achieved. The composition of the fermentable gas stream is illustrated by the below table.

| Compound | | |
|---|---|---|
| Oxygen | 3.1 | ppm |
| Acetylene | 0.960 | ppm |
| Hydrogen Cyanide | 0.280 | ppm |

Trace amounts of methane were detected in the fermentable gas stream. However, the amount of methane in the outlet stream was similar to the amount of methane detected as an impurity in the inlet stream, thus no production of methane was detected. Trace ethane and ethylene were detected. Ethane and ethylene are products from acetylene removal and are not microbial inhibitors. Acetaldehyde was detected in the fermentable gas stream. Acetaldehyde is not a microbial inhibitor. No other impurities were detected in the outlet stream using this configuration. No microbial inhibitors were formed using this configuration of beds.

The outlet concentration of the CO was 15.9% by volume. This outlet concentration corresponds to 3.0% consumption of the input CO, which is well below a maximum consumption of 10%.

It will be appreciated that the foregoing examples, given for purposes of illustration, are not to be construed as limiting the scope of this disclosure. Although only a few exemplary embodiments of this disclosure have been described in detail above, those skilled in the art will readily appreciate that many variations of the theme are possible in the exemplary embodiments without materially departing from the novel teachings and advantages of this disclosure. Accordingly, all such modifications are intended to be included within the scope of this disclosure that is defined in the following claims and all equivalents thereto. Further, it is recognized that many embodiments may be conceived in the design of a given system that do not achieve all of the advantages of some embodiments, yet the absence of a particular advantage shall not be construed to necessarily mean that such an embodiment is outside the scope of the present disclosure.

Although the foregoing text sets forth a detailed description of numerous different embodiments of the disclosure, it should be understood that the scope of the disclosure is defined by the words of the claims set forth at the end of this patent. The detailed description is to be construed as exemplary only and does not describe every possible embodiment of the disclosure because describing every possible embodiment would be impractical, if not impossible. Numerous alternative embodiments could be implemented, using either current technology or technology developed after the filing date of this patent, which would still fall within the scope of the claims defining the disclosure.

Thus, many modifications and variations may be made in the techniques and structures described and illustrated herein without departing from the spirit and scope of the present disclosure. Accordingly, it should be understood that the methods and apparatus described herein are illustrative only and are not limiting upon the scope of the disclosure.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgment that that prior art forms part of the common general knowledge in the field of endeavor in any country.

The use of the terms “a” and “an” and “the” and similar referents in the context of describing the invention (especially in the context of the following claims) are to be construed to cover both the singular and the plural unless otherwise indicated herein or clearly contradicted by context. The terms “comprising,” “having,” “including,” and “containing” are to be construed as open-ended terms (i.e., meaning “including, but not limited to”) unless otherwise noted. The term “consisting essentially of” limits the scope of a composition, process, or method to the specified materials or steps, or to those that do not materially affect the basic and novel characteristics of the composition, process, or method. The use of the alternative (i.e., “or”) should be understood to mean either one, both, or any combination thereof of the alternatives. As used herein, the term “about” means 20% of the indicated range, value, or structure, unless otherwise indicated. As used herein, the term “substantially” means to a great extent or degree and having significance.

Recitation of ranges of values herein is merely intended to serve as a shorthand method of referring individually to each separate value falling within the range, unless otherwise indicated herein, and each separate value is incorporated into the specification as if it were individually recited herein. For example, any concentration range, percentage range, ratio range, integer range, size range, or thickness range is to be understood to include the value of any integer within the recited range and, when appropriate, fractions thereof (such as one tenth and one hundredth of an integer), unless otherwise indicated. Unless otherwise stated, percentages are mass-%.

All methods described herein can be performed in any suitable order unless otherwise indicated herein or otherwise clearly contradicted by context. The use of any and all examples, or exemplary language (i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Select embodiments of this invention are described herein. Variations of those embodiments may become apparent to those of ordinary skill in the art upon reading the foregoing description. It is expected that skilled artisans to employ such variations as appropriate, and it is intended for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Unless the context dictates the contrary, all ranges set forth herein should be interpreted as being inclusive of their endpoints, and open-ended ranges should be interpreted to include commercially practical values. Similarly, all lists of values should be considered as inclusive of intermediate values unless the context indicates the contrary.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

It must be noted that as used herein and in the appended claims, the singular forms “a”, “an”, and “the” include plural referents unless the context clearly dictates otherwise.

Unless the context requires otherwise, throughout this description and the claims, the words “comprise”, “comprising”, and the like, are to be construed in an inclusive sense as opposed to an exclusive or exhaustive sense, that is to say, in the sense of including, but not limited to.

### Embodiments

Embodiment 1. A process for removing impurities from an input gas stream to produce a fermentable gas stream comprising:
a. heating the input gas stream to a temperature that is effective for a deoxygenation catalyst and below a reduction temperature of a sulfur guard bed material to generate a heated input gas stream;
b. contacting, in a vessel, the heated input gas stream with:
   i. a hydrolysis catalyst bed to remove hydrogen cyanide to less than 1 ppm hydrogen cyanide and/or carbonyl sulfide to less than 1 ppm carbonyl sulfide;
   ii. a sulfur guard bed containing material effective to remove and/or react sulfur-containing compounds located concurrent with or downstream of the hydrolysis bed; and
   iii. a deoxygenation catalyst bed located downstream of the sulfur guard bed;
c. wherein the fermentable gas stream comprises less than 100 ppm oxygen, less than 1 ppm acetylene, and less than 1 ppm hydrogen cyanide.

Embodiment 2. The process of embodiment 1 further comprising contacting, in the vessel, the heated input gas stream with a hydrocarbon removal adsorbent bed located upstream of the hydrolysis catalyst bed or contacting the input gas stream, in a module upstream of the heating and the vessel, with a hydrocarbon removal adsorbent bed.

Embodiment 3. The process of embodiment 1 or 2 wherein the hydrolysis catalyst bed and the sulfur guard bed are a physical mixture forming a combined hydrolysis and sulfur guard bed.

Embodiment 4. The process of any of embodiments 1 to 3 wherein the hydrolysis catalyst bed and the sulfur guard bed are a physical mixture forming a combined hydrolysis and sulfur guard bed.

Embodiment 5. The process of any of embodiments 1 to 4 further comprising passing at least a portion of the fermentable gas stream to a bioreactor, wherein the bioreactor contains a culture comprising a fermentation broth and at least one C1-fixing microorganism.

Embodiment 6. The process of any of embodiments 1 to 5 wherein at least a portion of the input gas stream is a synthesis gas and/or a producer gas.

Embodiment 7. The process of any of embodiments 1 to 6 wherein the process further comprises measuring the concentration of impurities in the input gas stream and/or the fermentable gas stream.

Embodiment 8. The process of any of embodiments 1 to 7 wherein the input gas stream comprises CO, CO₂, H₂, or any combination thereof.

Embodiment 9. The process of any of embodiments 1 to 8 wherein the sulfur guard bed material is zinc oxide or copper and zinc oxide supported on alumina.

Embodiment 10. The process of any of embodiments 1 to 9 wherein the deoxygenation catalyst comprises copper supported on alumina, silica, titania, ceria, lanthana, carbon, silica-alumina, or a zeolite.

Embodiment 11. The process of any of embodiments 1 to 10 wherein the hydrocarbon removal adsorbent is activated carbon.

Embodiment 12. The process of any of embodiments 1 to 11 wherein the hydrolysis bed and the sulfur guard bed are a combined hydrolysis and sulfur guard bed comprising bifunctional zinc oxide on alumina support or copper and zinc oxide on alumina support.

Embodiment 13. The process of any of embodiments 1 to 12 wherein the hydrolysis bed and the sulfur guard bed are a combined hydrolysis and sulfur guard bed comprising bifunctional zinc oxide on alumina support or copper and zinc oxide on alumina support.

Embodiment 14. An apparatus comprising:
a. a heating device having a heating device gas inlet and a heating device gas outlet;
b. a vessel having a vessel gas inlet in fluid communication with the heating device gas outlet and a vessel fermentable gas outlet wherein the vessel contains at least three beds comprising:
   i. a hydrolysis catalyst bed comprising alumina;
   ii. a sulfur guard bed comprising zinc oxide or copper and zinc oxide on an alumina support located concurrent with or downstream of the hydrolysis bed;
   iii. a deoxygenation catalyst bed located downstream of the sulfur guard bed, wherein the deoxygenation catalyst comprises copper supported on alumina, silica, titania, ceria, lanthana, carbon, silica-alumina, or zeolites; and
c. a bioreactor having a bioreactor gas inlet in fluid communication with the vessel fermentable gas outlet and a bioreactor fermentation broth output wherein the bioreactor comprises at least one C1-fixing microorganism.

Embodiment 15. The apparatus of embodiment 14 further comprising at least one monitoring device in communication with the vessel gas inlet, the vessel fermentable gas outlet, or both.

Embodiment 16. The apparatus of any of embodiments 14 to 15 wherein the vessel further comprises a hydrocarbon removal bed comprising activated charcoal located in the vessel upstream of the hydrolysis catalyst bed.

Embodiment 17. The apparatus of any of embodiments 14 to 16 further comprising a hydrocarbon removal module comprising activated charcoal and having a hydrocarbon removal module gas inlet and hydrocarbon removal module gas outlet, the hydrocarbon removal module gas outlet in fluid communication with the heating device gas inlet.

Embodiment 18. A process of revamping a gas treatment system comprising:
a. co-locating, in a repurposed vessel,
   i. a hydrolysis catalyst bed to remove hydrogen cyanide to less than 1 ppm hydrogen cyanide and/or carbonyl sulfide to less than 1 ppm carbonyl sulfide;
   ii. a sulfur guard bed containing material effective to remove and/or react sulfur-containing compounds located concurrent with or downstream of the hydrolysis bed; and
   iii. a deoxygenation catalyst bed located downstream of the sulfur guard bed.

Embodiment 19. The process of embodiment 18 further comprising connecting a repurposed heating device upstream of the repurposed vessel.

Embodiment 20. The process of any of embodiments 18 to 19 further comprising connecting a hydrocarbon removal module comprising activated charcoal upstream of the repurposed heating device.

Embodiment 21. A process for removing impurities from an input gas stream to produce a fermentable gas stream comprising:
a. heating the input gas stream to a temperature that is effective for a deoxygenation catalyst and below a reduction temperature of a sulfur guard bed material to generate a heated input gas stream;
b. contacting, in a first vessel, the heated input gas stream with:
   i. a hydrolysis catalyst bed to remove hydrogen cyanide to less than 1 ppm hydrogen cyanide and/or carbonyl sulfide to less than 1 ppm carbonyl sulfide;
   ii. a first sulfur guard bed containing material effective to remove and/or react sulfur-containing compounds, the first sulfur guard bed located concurrent with or downstream of the hydrolysis bed;
c. contacting, in a second vessel, an effluent of the first vessel with:
   i. a second sulfur guard bed containing material effective to remove and/or react sulfur-containing compounds,
   ii. a deoxygenation catalyst bed located downstream of the sulfur guard bed;
d. wherein the fermentable gas stream comprises less than 100 ppm oxygen, less than 1 ppm acetylene, and less than 1 ppm hydrogen cyanide.

Embodiment 22. The process of claim 21 further comprising contacting, in the vessel, the heated input gas stream with a hydrocarbon removal adsorbent bed located upstream of the hydrolysis catalyst bed or contacting the input gas stream, in a module upstream of the heating and the vessel, with a hydrocarbon removal adsorbent bed.

Embodiment 23. The process of embodiment 21 wherein the hydrolysis catalyst bed and the first sulfur guard bed are a physical mixture forming a combined hydrolysis and sulfur guard bed.

Embodiment 24. The process of any of embodiments 21 to 23 wherein the hydrolysis catalyst bed and the first sulfur guard bed are a physical mixture forming a combined hydrolysis and sulfur guard bed.

Embodiment 25. The process of any of embodiments 21 to 24, further comprising passing at least a portion of the fermentable gas stream to a bioreactor, wherein the bioreactor contains a culture comprising a fermentation broth and at least one C1-fixing microorganism.

Embodiment 26. The process of any of embodiments 21 to 25, wherein at least a portion of the input gas stream is a synthesis gas and/or a producer gas.

Embodiment 27. The process of any of embodiments 21 to 26, wherein the process further comprises measuring the concentration of impurities in the input gas stream and/or the fermentable gas stream.

Embodiment 28. The process of any of embodiments 21 to 27, wherein the input gas stream comprises CO, CO₂, H₂, or any combination thereof.

Embodiment 29. The process of any of embodiments 21 to 28, wherein the first and or second sulfur guard bed material is zinc oxide or copper and zinc oxide supported on alumina.

Embodiment 30. The process of any of embodiments 21 to 29 wherein the deoxygenation catalyst comprises copper supported on alumina, silica, titania, ceria, lanthana, carbon, silica-alumina, or a zeolite.

Embodiment 31. The process of any of embodiments 21 to 30 wherein the hydrocarbon removal adsorbent is activated carbon.

Embodiment 32. The process of any of embodiments 21 to 31 wherein the hydrolysis bed and the first sulfur guard bed are a combined hydrolysis and first sulfur guard bed comprising bifunctional zinc oxide on alumina support or copper and zinc oxide on alumina support.

Embodiment 33. The process of any of embodiments 21 to 32 wherein the hydrolysis bed and the first sulfur guard bed are a combined hydrolysis and first sulfur guard bed comprising bifunctional zinc oxide on alumina support or copper and zinc oxide on alumina support.

Embodiment 34. An apparatus comprising:
a. a heating device having a heating device gas inlet and a heating device gas outlet;
b. a first vessel having a first vessel gas inlet in fluid communication with the heating device gas outlet and a first vessel gas outlet wherein the first vessel contains at least two beds comprising:
   i. a hydrolysis catalyst bed comprising alumina;
   ii. a first sulfur guard bed comprising zinc oxide or copper and zinc oxide on an alumina support located concurrent with or downstream of the hydrolysis bed;
c. a second vessel having a second vessel gas inlet in fluid communication with the first vessel gas outlet and a second vessel fermentable gas outlet wherein the second vessel contains at least two beds comprising:
   i. a second sulfur guard bed comprising zinc oxide or copper and zinc oxide on an alumina support;
   ii. a deoxygenation catalyst bed located downstream of the second sulfur guard bed, wherein the deoxygenation catalyst comprises copper supported on alumina, silica, titania, ceria, lanthana, carbon, silica-alumina, or zeolites; and
d. a bioreactor having a bioreactor gas inlet in fluid communication with the second vessel fermentable gas outlet and a bioreactor fermentation broth output wherein the bioreactor comprises at least one C1-fixing microorganism.

Embodiment 35. The apparatus of embodiment 34 further comprising at least one monitoring device in communication with the vessel gas inlet, the vessel fermentable gas outlet, or both.

Embodiment 36. The apparatus of embodiments 34 or 35 wherein the vessel further comprises a hydrocarbon removal bed comprising activated charcoal located in the vessel upstream of the hydrolysis catalyst bed.

Embodiment 37. The apparatus of any of embodiments 34 to 36 further comprising a hydrocarbon removal module comprising activated charcoal and having a hydrocarbon removal module gas inlet and hydrocarbon removal module gas outlet, the hydrocarbon removal module gas outlet in fluid communication with the heating device gas inlet.

Embodiment 39. A process of revamping a gas treatment system comprising:
a. co-locating, in a first repurposed vessel,
   i. a hydrolysis catalyst bed to remove hydrogen cyanide to less than 1 ppm hydrogen cyanide and/or carbonyl sulfide to less than 1 ppm carbonyl sulfide; and
   ii. a first sulfur guard bed containing material effective to remove and/or react sulfur-containing compounds located concurrent with or downstream of the hydrolysis bed;
b. co-locating in a second repurposed vessel.
   i. a second sulfur guard bed containing material effective to remove and/or react sulfur-containing compounds; and
   ii. a deoxygenation catalyst bed located downstream of the second sulfur guard bed.

Embodiment 39. The process of embodiment 38 further comprising connecting a repurposed heating device upstream of the repurposed vessel.

Embodiment 40. The process of embodiments 38 or 39 further comprising connecting a hydrocarbon removal module comprising activated charcoal upstream of the repurposed heating device.

1. A process for removing impurities from an input gas stream to produce a fermentable gas stream comprising less than about 100 ppm oxygen, less than about 1 ppm acetylene, and less than about 1 ppm hydrogen cyanide, the process comprising:
a. heating the input gas stream to a temperature that is effective for a deoxygenation catalyst and below a reduction temperature of a sulfur guard bed material to generate a heated input gas stream;
b. contacting the heated input gas stream with:
a hydrolysis catalyst bed to remove hydrogen cyanide to less than 1 ppm hydrogen cyanide and/or carbonyl sulfide to less than 1 ppm carbonyl sulfide;
one or more optional sulfur guard beds containing material effective to remove and/or react sulfur-containing compounds located concurrent with or downstream of the hydrolysis bed;
and a deoxygenation catalyst bed located downstream of the one or more optional sulfur guard beds; and
wherein the hydrolysis bed, the one or more optional sulfur guard beds, and the deoxygenation catalyst bed are contained within a single vessel, or wherein the hydrolysis bed and a first optional sulfur guard bed are contained within a first vessel, and a second optional guard bed and the deoxygenation catalyst bed are contained within a second vessel.

2. The process of claim 1 further comprising contacting, in the single vessel or in the first vessel, the heated input gas stream with a hydrocarbon removal adsorbent bed located upstream of the hydrolysis catalyst bed or contacting the input gas stream with a hydrocarbon removal adsorbent bed contained in a module upstream of the heating of the input gas stream.

3. The process of claim 1 wherein the hydrolysis catalyst bed and the sulfur guard bed or the first sulfur guard bed are a physical mixture forming a combined hydrolysis and sulfur guard bed.

4. The process of claim 1, further comprising passing at least a portion of the fermentable gas stream to a bioreactor, wherein the bioreactor contains a culture comprising a fermentation broth and at least one C1-fixing microorganism.

5. The process of claim 1, wherein at least a portion of the input gas stream is a synthesis gas and/or a producer gas.

6. The process of claim 1, wherein the process further comprises measuring the concentration of impurities in the input gas stream and/or the fermentable gas stream.

7. The process of claim 1, wherein the input gas stream comprises CO, CO₂, H₂, or any combination thereof.

8. The process of claim 1 wherein the sulfur guard bed material in any sulfur guard bed is zinc oxide or copper and zinc oxide supported on alumina.

9. The process of claim 1 wherein the deoxygenation catalyst comprises copper supported on alumina, silica, titania, ceria, lanthana, carbon, silica-alumina, or a zeolite.

10. The process of claim 1 wherein the hydrocarbon removal adsorbent is activated carbon.

11. The process of claim 1 wherein the hydrolysis bed and the sulfur guard bed or the first sulfur guard bed are a combined hydrolysis and sulfur guard bed comprising bifunctional zinc oxide on alumina support or copper and zinc oxide on alumina support.

12. An apparatus comprising:
a heating device having a heating device gas inlet and a heating device gas outlet;
a single vessel having a single vessel gas inlet in fluid communication with the heating device gas outlet and a single vessel fermentable gas outlet wherein the single vessel contains at least three beds comprising:
a hydrolysis catalyst bed comprising alumina;
a sulfur guard bed comprising zinc oxide or copper and zinc oxide on an alumina support located concurrent with or downstream of the hydrolysis bed;
a deoxygenation catalyst bed located downstream of the sulfur guard bed, wherein the deoxygenation catalyst comprises copper supported on alumina, silica, titania, ceria, lanthana, carbon, silica-alumina, or zeolites;
or
a first vessel having a first vessel gas inlet in fluid communication with the heating device gas outlet and a first vessel gas outlet wherein the first vessel contains at least two beds comprising:
a hydrolysis catalyst bed comprising alumina;
a first sulfur guard bed comprising zinc oxide or copper and zinc oxide on an alumina support located concurrent with or downstream of the hydrolysis bed; and
a second vessel having a second vessel gas inlet in fluid communication with the first vessel gas outlet and a second vessel fermentable gas outlet wherein the second vessel contains at least two beds comprising:
a second sulfur guard bed comprising zinc oxide or copper and zinc oxide on an alumina support;
a deoxygenation catalyst bed located downstream of the second sulfur guard bed, wherein the deoxygenation catalyst comprises copper supported on alumina, silica, titania, ceria, lanthana, carbon, silica-alumina, or zeolites;
and
a bioreactor having a bioreactor gas inlet in fluid communication with the single vessel fermentable gas outlet or the second vessel fermentable gas outlet and the bioreactor having a bioreactor fermentation broth output wherein the bioreactor comprises at least one C1-fixing microorganism.

13. The apparatus of claim 12 further comprising at least one monitoring device in communication with the single vessel gas inlet or the first vessel gas inlet, the vessel fermentable gas outlet or the second vessel fermentable gas outlet, or both.

14. The apparatus of claim 12 wherein the single vessel or the first vessel further comprises a hydrocarbon removal bed comprising activated charcoal located in the single vessel or the first vessel upstream of the hydrolysis catalyst bed.

15. The apparatus of claim 12 further comprising a hydrocarbon removal module comprising activated charcoal and having a hydrocarbon removal module gas inlet and hydrocarbon removal module gas outlet, the hydrocarbon removal module gas outlet in fluid communication with the heating device gas inlet.

16. A process of revamping a gas treatment system comprising:
a. co-locating
i. a hydrolysis catalyst bed to remove hydrogen cyanide to less than 1 ppm hydrogen cyanide and/or carbonyl sulfide to less than 1 ppm carbonyl sulfide;
ii. an optional sulfur guard bed containing material effective to remove and/or react sulfur-containing compounds located concurrent with or downstream of the hydrolysis bed; and
iii. a deoxygenation catalyst bed located downstream of the sulfur guard bed;
in a single repurposed vessel;
or
b. co-locating,
i. a hydrolysis catalyst bed to remove hydrogen cyanide to less than 1 ppm hydrogen cyanide and/or carbonyl sulfide to less than 1 ppm carbonyl sulfide; and
ii. a first sulfur guard bed containing material effective to remove and/or react sulfur-containing compounds located concurrent with or downstream of the hydrolysis bed;
in a first repurposed vessel, and
c. co-locating
i. a second sulfur guard bed containing material effective to remove and/or react sulfur-containing compounds; and
ii. a deoxygenation catalyst bed located downstream of the second sulfur guard bed;
in a second repurposed vessel.

17. The process of claim 16 further comprising connecting a repurposed heating device upstream of the single repurposed vessel or the first repurposed vessel.

18. The process of claim 17 further comprising connecting a hydrocarbon removal module comprising activated charcoal upstream of the repurposed heating device.
